(19) United States
(12) Patent Application Publication
(10) Pub. No.: US 2005/0150160 A1
(43) Pub. Date: Jul. 14, 2005

(54) Method for selecting crop varieties

(76) Inventors: Daniel G. Norgaard, Sibley, IA (US); Bruce C. Burns, Heron Lake, MN (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 10/974,478

(22) Filed: Oct. 27, 2004

Related U.S. Application Data
(60) Provisional application No. 60/514,954, filed on Oct. 28, 2003.

(51) Int. Cl. A01H 5/00

(52) U.S. Cl. 47/58.1 SE

(57) ABSTRACT

A process of recommending crop varieties based on management categories. The management categories are determined by utilizing indices, which measure the economic implication of physical and chemical properties of a specific soil type in a region. The management categories may then be indicated graphically by indicia overlaying digitized soil maps, each of the indicia grouping soil types within a management category. Each crop variety is characterized by how the crop variety performs in each of the management categories. Each of the crop varieties may then be assigned an indicium of the management category for which it is adapted.

### CROSS-REFERENCES TO RELATED APPLICATIONS

This application claims priority under 35 U.S.C. § 119 (e) to, and hereby incorporates by reference, U.S. Provisional Application No. 60/514,954, filed 28 Oct. 2003.

### BACKGROUND OF THE INVENTION

1. Field of the Invention

This invention relates to methods for crop varietal selection and, in particular, this invention relates to methods for crop varietal selection which utilize soil characteristic indices and adaptive varietal characteristics in an integrated data base.

2. Background of the Invention

The set of crop varieties selected and used by a farmer determines, in large part, the potential yield that can be realized for a growing season. Hence, selecting the most suitable and productive crop varieties is an ongoing issue confronting farmers at the beginning of each growing season. Crop varieties are known to be specifically adapted, inter alia, by maturity, by tolerance to specific soil types and soil moisture availability, by reactions to pests, and by suitability to various tillage practices. Typically, farmers select specific varieties based on information provided from discrete, unassociated sources such as seed companies, extension services, prior experience, and peer recommendations. Using the foregoing information, farmers often select one or more crop varieties to be planted in a specific field. However, even if more than one variety is planted in a given field, each variety is typically planted the entire length or width of the field. Because fields almost always contain more than one soil type, planting a single variety, or set of varieties, in an entire field often results in planting less than optimally adapted varieties to appreciable portions of a given field. To date, no single and comprehensive system or method has enabled farmers to select crop varieties using a database with information such as crop varietal response to soil type, anticipated precipitation (rainfall and/or irrigation), reaction to pests (diseases, insects, toxic mineral levels in the soil solution), anticipated levels of crop nutrients, and the like. Moreover, no currently available integrated system utilizes agronomically important characteristics from a specific field to enable farmers to select and plant specific crop varieties in varying soil types present within a given field. Additionally, no currently available system uses indicia, such as colors, to characterize both the soil types and varieties available, the varieties adapted to a given soil type having the same indicium as the soil type itself.

### SUMMARY OF THE INVENTION

This invention substantially meets these needs by providing an integrated data set advantageous for choosing crop varieties and for other uses such as nutrient management as well. The data set includes information derived from physical characteristics of the soils in a region and information characterizing crop varietal adaptation to these physical characteristics. The physical characteristics of the soils include slope, degree or extent of erosion, typical crop (e.g., corn and soybean) yields, types and proportions of soil particles present (texture), pH, solum, water holding capacity, permeability, presence of saturated regions within soil types, soluble salts, calcium carbonate equivalent, organic matter, and cation exchange capacity. The physical characteristics are utilized to provide indices related to crop growth and development such as slope index, eroded soil index, water table index, soil permeability index, root zone drainage index, soil texture index, available water capacity index, soil reaction index, iron chlorosis severity index, Phytophthora/fungi potential index, organic matter index, and cation exchange capacity index. These indices group soil types into sets which respond to common management regimes and in which a given set of crop varieties is adapted. The foregoing indices are then used to place soil types into management categories, which may be graphically depicted by a digitized soils map. These management categories further group soil types within which similar management regimes and crop varieties can be used. Exemplary management categories include those for crop (e.g., corn, soybeans) placement and nutrient (e.g., N, P, K, Zn, S, pH (lime)) management. A given set of available crop varieties is characterized by its adaptation to each of the relevant management categories. Each soil management category and each set of crop varieties adapted to be grown in soils in the soil management category is designated by a unique common indicium such as a color.

There is provided a process for selecting a crop variety. The process may include viewing a digitized map of a field, the digitized map depicting areas in a field denoted by an indicium (e.g., a color) having optimized adaptation for a set of crop varieties. The process may further include selecting the crop variety from the variety set, each variety in the variety set denoted by the same indicium.

There is also provided a process of integrating a set of soil characteristics and a set of crop variety characteristics. The process may include 1) obtaining a set of properties (e.g., the foregoing physical properties) characterizing a subset of soil types; 2) using the set of properties to obtain a set of corresponding indices; 3) using the set of indices to obtain a set of management categories; 4) obtaining a set of adaptation characteristics for a corresponding set of crop varieties; and 5) using the set of adaptation characteristics to designate which of the set of crop varieties should be planted in each of the management categories.

There is yet provided a process for determining crop management categories, which may include compiling a set of physical properties for a set of soil types in a region and assigning each of the soil types in the region to one of the crop management categories.

There is still yet provided a process of determining a seeding rate for a variety to be seeded, the process including 1) providing a planter with a variable seeding rate, the planter in electrical or electromagnetic communication with a digitized soil map, the digitized soil map having areas defined by crop management categories; and seeding the variety be seeding rate of the variety determined by the position of the planter relative to the crop management categories.

In one embodiment, management (e.g., corn seed placement) categories are, in part, determined by soil water holding capacity as measured by a plurality of indices.

In another embodiment, management (e.g., soybean seed placement) categories are determined by predisposition of the soil types therein and varieties to Phytophthora.

In yet another embodiment, management (e.g., soybean seed placement) categories are determined by predisposition of the soil types therein and varieties to iron chlorosis.

In still yet another embodiment, management categories for corn and/or soybeans are utilized for nutrient management.

In yet still another embodiment, specific crop varieties can be separately planted to each management category.

It is a feature of the invention to provide an integrated data set for selecting a crop variety, the integrated set based on objective measures of physical and agronomic properties of soil types in a region.

It is an advantage of the foregoing feature that the information used to select crop varieties is based on reliable objective data.

It is another feature of the invention to provide a common indicium for a management category and for crop varieties adapted to soils represented by the indicium.

It is an advantage of the foregoing feature that choosing one or more crop varieties is simplified by using the common indicium.

These and other features and advantages of this invention will become apparent from the description which follows, when considered in view of the accompanying drawings.

### BRIEF DESCRIPTION OF THE DRAWINGS

FIG. 1 is a depiction of a digitized soil map showing areas characterized by corn management categories;

FIG. 2 is a depiction of a digitized soil map showing areas with common degrees of predisposition for iron chlorosis;

FIG. 3 is a depiction of a digitized soil map showing areas with common degrees of predisposition for Phytophthora infection.

FIG. 4 shows exemplary indicia for crop nutrient management categories.

FIG. 5 shows exemplary corn hybrids grouped by the present management categories;

FIG. 6 shows exemplary soybean varieties grouped by the present management categories;

FIG. 7 is a flow chart depicting a method of classifying a predominant soil type into Corn Seed Placement Categories according to the invention;

FIG. 8 is a flow chart depicting a method of classifying a predominant soil type into Soybean Phytophthora Soybean Seed Placement Categories according to the invention;

FIG. 9 depicts a computer screen with permeability indices of soil types present in a field;

FIG. 10 depicts a computer screen with soybean Phytophthora Soybean Seed Placement Categories in a portion of a field, a portion of the screen displaying recommended soybean varieties for the field and placement categories; and

FIG. 11 shows a computer screen with soybean Phytophthora Soybean Seed Placement Categories in a field.

It is understood that the above-described figures are merely illustrative of the present invention and do not limit the scope thereof.

The patent or application file contains at least one drawing executed in color. Copies of this patent or patent application publication with color drawings will be provided by the Office upon request and payment of the necessary fee.

### DETAILED DESCRIPTION

Each of the features and methods disclosed may be utilized separately or in conjunction with other features and methods either disclosed herein or known to persons of ordinary skill in the art to provide improved methods for selecting crop varieties. Representative examples of the teachings of the present invention, the examples utilizing many of these methods, will now be described in detail. This detailed description is merely intended to teach a person of skill in the art further details for practicing various aspects of the present teachings and is not intended to limit the scope of the invention. Therefore, combinations of features and methods disclosed in the following detailed description may not be necessary to practice the invention in the broadest sense and are instead taught merely to particularly describe representative and preferred embodiments of the invention.

Unless otherwise defined, all technical and scientific terms used herein have the same meaning as commonly understood by one of ordinary skill in the art to which this invention belongs. However, in case of conflict, the present specification, including express or implied definitions, will control. Although methods and materials similar or equivalent to those described herein can be used to practice the invention, suitable methods and materials are described below. All patent applications, issued patents, and other documents mentioned herein are incorporated by reference in their entirety.

All or some of the soil characteristics using in the present method may be obtained from digitized field maps. Digitized field maps, in turn, may be generated by accessing information obtained from, e.g., aerial mapping protocols. In one exemplary method of generating digitized field maps, U.S. Pat. No. 5,467,271, issued 14 Nov. 1995 to Abel et al., discloses a mapping and analysis system, which generates and analyzes agricultural maps to match farm inputs of a farm field to current soil and vegetation characteristics to optimize the productivity of the field. The mapping and analysis system includes an air-based device for generating spectral image data related to at least one of vegetation stress and a soil characteristic for a portion of the field. A position device generates position data related to the position of the air-based device with respect to the portion of the farming field. A georeferencing device using, e.g., GPS and LORAN, synchronizes the position data with the spectral image data to generate georeferenced spectral image data. A database is generated using the georeferenced data to monitor and analyze the farming field for a growing season to improve productivity thereof.

Using digital maps in a field to control seeding rates, fertilizer rates, and to vary the variety being seeded and the blend of fertilizer (or other treatments) being applied requires one to locate one's position precisely in a given field. To this end, U.S. Pat. No. 6,397,147, issued 28 May 2002 to Whitehead, discloses a technique of accurately determining the relative position between two points, in real-time, using a single GPS receiver that makes measurements of signals transmitted from GPS satellites. A technique is applied where differential correction terms are computed as a location at an instant of time, and then applied to further times, after applying atmospheric delay adjustments, so that the position of the GPS receiver is determined accurately relative to the position at the original instant of time.

In another example of position determining technology, U.S. Pat. No. 6,570,534, issued 27 May 2003 to Cohen et al., discloses a low-cost, solid-state position sensor system suitable for making precise code and carrier phase measurements in the L1 and L2 bands of GPS. The system uses an ordinary, low-cost OEM card single-frequency carrier phase tracking C/A code receiver and includes low-cost hardware for sensing the L1 and L2 components of GPS carrier phase. Such measurements are suitable for general use in a variety of fields, including surveying. They are also of sufficient quality to be used in controlling heavy machinery, such as aircraft, farm tractors, and construction and mining equipment. A C/A code continuous tracking GPS receiver is used to produce GPS positioning fixes and real-time L1 carrier phase measurements. This C/A code receiver generates timing and reference information for a digital sampling component. This sampling component processes the L1 and L2 signals from the GPS signals. A digital signal-processing component coupled to this sampling component processes the raw samples in synchronous, batch form including a step to precisely unwrap the P (Y) carrier phase to baseband. The receiver outputs synchronous, carrier phase measurements associated with each ranging source and signal observable. The synchronous raw carrier phase measurements from the continuous tracking C/A code receiver and the digital sampling component may be used to resolve the cycle ambiguities to each ranging source with respect to a reference station at a known location. Within a short interval typically tens of seconds from initial turn on, continuous, synchronous raw measurements are provided by the GPS receiver and processed into precise position fixes.

After being generated, digitized soil maps and precise position locating technology may be utilized to enable precision fertilizer delivery. The amount and composition of the fertilizer applied may be varied to accommodate needs determined by digitized soil maps of fields to which the fertilizer is applied. For example, U.S. Pat. No. 4,630,773, issued 23 Dec. 1986 to Ortlip, discloses a fertilizer spreading apparatus, which includes a vehicle carrying a plurality of product bins, each for carrying a different fertilizing product. Feeder devices are provided to meter product from the bins which is collected and spread over the field to be fertilized. A computerized control system is provided which holds a digital soil map of the location of various soil types in the field to be fertilized. The computerized control system is responsive to vehicle locating technology, e.g., a LORAN locater unit, for determining the location of the vehicle in the field, looking up the type of soil the vehicle is positioned currently over based on its location, and adjusting feeder operation in response thereto.

Once gathered, agronomic information must be associated with the characteristics recorded by digitized soil maps. To this end, U.S. Patent Application Publication 2002/0022929, published 21 Feb. 2002 and listing Ell as inventor, discloses a system and method for creating field attribute maps for site-specific farming. The field attribute maps contain agricultural data collected from a field and converted into a format used to create application maps. To create field attribute maps, agricultural data is collected from a field and input to a mapping system. The agricultural data is then cleansed and validated. The cleansing process corrects any data errors and converts the data into a standard format. The validation process verifies the latitude and longitude of the data. The data is then converted into a two-dimensional grid format. The end result is a field broken into multiple grid cells, each cell containing agricultural data. The two-dimensional grid format allows the mapping system to more efficiently create application maps.

Digitized soil maps may also be used, inter alia, to control the population of seeds planted in fields. For example, U.S. Pat. No. 5,646,846, issued 8 Jul. 1997 to Bruce et al., discloses a global positioning planter system for planting seeds by a planter. A population control controls the amount of seed dispensed by the planter during seeding. A global positioning system computer with digitized maps connects to the population control. The population control connects to the planter for planting seeds such as corn or beans or to seed drills for controlling the seed drill, and plants the seeds according to the population control which receives and transmits data with the global positioning system computer, the seed population being varied according to the digitized soil map.

Applying fertilizer or seeding with rates responsive to soil characteristics requires that the farm implement be located precisely in a field. To this end, U.S. Pat. Nos. 5,684,476 and 5,955,973, issued 4 Nov. 1997 and 21 Sep. 1999, respectively, to Anderson, disclose a location system used in a vehicle moving within an area at a selected speed and in a selected direction. A heading sensor provides a heading signal representing the direction of movement of the vehicle. A speed sensor provides a speed signal based on available reference signals representing the speed of the vehicle. A storage device stores initial position data representing a selected initial position of the vehicle and checkpoint data representing a navigation checkpoint location. A database stores a plurality of records. Each record includes geographic information data representing selective aspects of the area. A processor estimates a current position signal representing an estimated current position of the vehicle based on values of the heading signal, values of the speed signal, the initial position signal, and on previous values of the current position signal. Values of the current position signal correspond to records stored in the database. A correction device selectively corrects the current position signal based on selected position inputs, which indicate an approximate vehicle position relative to the navigation checkpoint location. An alerting device obtains an alerting signal indicating that the vehicle has reached a selected region within the area based on the current position signal and the geographic information data.

Different crop varieties can also be seeded in different parts of the field by using digitized soil maps and position locating equipment and protocols. For example, U.S. Pat. Nos. 5,913,914 and 5,913,915, each issued 22 Jun. 1999 to McQuinn, disclose an improved mobile agricultural products application system including a multi-variable rate dispensing system particularly adaptable for use in site-specific farming. Selected discrete crop input delivery information unique to selected on-board crop input storage devices, and/or crop input transport systems, and/or crop input dispensing points is combined with anticipated field reference point data obtained with a machine positioning system, e.g. “Dead Reckoning”, GPS, and/or radar, and a computer, to direct independent functioning of selected on-board storage devices, material transport systems, crop input release mechanisms and/or dispensing point mechanisms to ensure stored crop inputs are released and combined to vary a prescription of delivered crop inputs in a direction substantially transverse to the direction of machine travel as the crop input applicator machine(s) travels over a predetermined geographic land area. The improved system can selectively and exclusively accommodate precise application of seeds as to different rates and/or varieties of seeds at different points on a variable rate crop input applicator machine, or can optionally accommodate seed application in combination with other crop inputs. The multi-variable rate dispensing system provides environmental advantages to all through enhanced resource management by more accurately and precisely placing crop inputs resulting in a significant reduction in wasted resources.

In another example, U.S. Pat. No. 5,956,255, issued 21 Sep. 1999 to Flamme, discloses a performance monitor for a seed-planting implement. The monitor is preferably used with a planting system including a planting implement coupled to a tractor. The target rate at which seed is planted by the implement in the soil of an agricultural field is controlled based upon a control signal. The actual seed-planting rate is monitored using an optical seed sensor supported by the implement at a location where seed exits the implement. The implement and tractor include data busses linked to each other. Signals from the seed sensors are transmitted to a controller on the tractor via the busses. The controller applies a display signal to an electronic display located in the tractor cab to produce an image which an operator can view to determine the actual seed application rate. The image also shows the target seed application rate to allow the operator to compare actual and target rates to determine whether the implement needs to be adjusted or repaired to eliminate or reduce any deviation in rates. Seed application rates for each section of a multiple-section implement can be displayed sequentially for efficient use of the display, with the rate for each row unit also being displayed.

In yet another example, U.S. Pat. No. 6,024,035, issued 15 Feb. 2000 to Flamme, discloses a seed planter performance monitor. The monitor is used with a planting system including a planter coupled to a tractor. The target rate at which the planter deposits seeds into the soil is controlled with a control signal. The actual rate at which seeds are planted is monitored with an infrared seed sensor supported by the planter at the location where seeds exit the planter. The planter and tractor both include data busses. The signal from the seed sensor is transmitted to a controller on the tractor via the busses. The controller applies an appropriate signal to an electronic display in the cab of the tractor to produce an image thereon which an operator can view to determine the actual rate at which seeds are planted. The operator compares the target and the actual planting rates and adjusts or controls the planter to place the rates in general correspondence by varying planter parameters such as air flow, pressure in the planter, or brush spacing in the drum of the seed meter.

In still yet another example of using digitized soil maps to apply varying amounts of seed, fertilizer, and types of fertilizer blends, U.S. Pat. No. 6,122,581, issued 19 Sep. 2000 to McQuinn, discloses an improved mobile agricultural products application system including a multi-variable rate dispensing system adaptable for use in site-specific farming. Using this system, selected discrete crop input delivery information unique to selected on-board crop input storage devices, and/or crop input transport systems, and/or crop input dispensing points is combined with anticipated field reference point data obtained with a machine positioning system, e.g. “Dead Reckoning”, GPS, and/or radar, and a computer, to direct independent functioning of selected on-board storage devices, material transport systems, crop input release mechanisms and/or dispensing point mechanisms to ensure stored crop inputs are released and combined to vary a prescription of delivered crop inputs in a direction substantially transverse to the direction of machine travel as the crop input applicator machine(s) travels over a predetermined geographic land area. The system can selectively and exclusively accommodate precise application of seeds as to different rates and/or varieties of seeds at different points on a variable rate crop input applicator machine if so desired. The system can optionally accommodate seed application in combination with other crop inputs. The multi-variable rate dispensing system provides environmental advantages to all through enhanced resource management by more accurately and precisely placing crop inputs resulting in a significant reduction in wasted resources.

Digitized soil maps are also useful to characterize farm fields by soil types present, then to use the information obtained therefrom in a data network for purposes of identifying soil and crop treatments. The scope and type of treatments are at least partially determined by the data derived from the digitized soil maps. For example, U.S. Pat. No. 5,689,418, issued 18 Nov. 1997 to Monson, discloses an agricultural communications network including a master system which polls lower level systems for digital maps, each map comprising field character information indicative of a feature at each location of a farmer's field. An agronomist can correlate the data of the digital maps to ascertain common conditions which realize maximum yields.

Digitized soil maps and position determining equipment and protocols may also be used to gather information about crop productivity in relation to the soil characteristics contained in the digitized soil maps. To this end, U.S. Pat. No. 5,902,343, issued 11 May 1999 to Hale et al., discloses a field mapping system for an agricultural vehicle such as a combine, planter or cultivator. The system includes a circuit for determining the position of the vehicle relative to a field, and a sensor for sensing a characteristic (e.g., grain moisture content, grain harvest yield, soil compaction, altitude, etc.) at locations of the vehicle within the field. The system also includes an electronic display controlled by a control circuit coupled to the position determining circuit and the sensor. The control circuit applies signals to the electronic display which produces a map of the field including indicia of the characteristic at respective locations within the field. For example, if the characteristic is grain moisture content, different colors can be used on the display to represent different moisture levels. The signals are generated by the control circuit so that the portion of the field over which the characteristic is sampled is scaled to be displayed over substantially all of a portion of the display. Thus, as the area of the field which has been sampled increases, the scale of the displayed map is automatically rescaled to show all of the data.

In another example of gathering information about crop response to soil types, U.S. Pat. Nos. 6,029,106, issued 22 Feb. 2000, and 6,061,618, issued 9 May 2000, both to Hale et al., disclose a field mapping system for an agricultural vehicle such as a combine or tractor. The system includes a location signal generator for determining the position of the vehicle relative to a field, a correction signal generator for receiving correction signals used to improve the accuracy of the position determination and a sensing circuit for detecting a characteristic (e.g., grain moisture, grain flow, soil compaction, soil moisture) at predetermined locations of the vehicle within the field. The system also includes an electronic display controlled by a control circuit coupled to the location signal generator, the correction signal generator, and the sensing circuit. The control circuit applies signals to the electronic display which produces a map of the field which includes indicia of the characteristic at respective locations within the field. For example, if the characteristic is grain moisture, different colors can be used on the display to represent different moisture levels.

In yet another example, U.S. Patent Application Publication 2002/0035431, published 21 Mar. 2002 and listing Ell as inventor, discloses a system and method of creating application maps for site-specific farming and developed using a modular process. The first step of the process is to develop field attribute maps. The field attribute maps contain the various types of agricultural inputs used to create an application map. The second step of the process is to create crop input requirement maps. The crop input requirement maps combine the information from the field attribute maps and recommendation equations. The last step of the process is to create an application map. The application map combines the crop input requirement maps and product inputs to create a blend of commercial products to be applied to a field.

In still yet another example, U.S. Patent Application Publication 2002/0040273, published 4 Apr. 2002 and listing John et al. as inventors, discloses a software-based system and method for analyzing data contained in a computerized database. A plan document specifies data to be used by each of a plurality of software modules. A decision tree document identifies a set of the software modules to be invoked and specifies an order in which the identified set of software modules are to be invoked. Each of the identified set of software modules is provided a version of the plan document. Each version of the plan document provided to each of the identified set of software modules is transformed into a transformed plan document such that each one of the identified sets of software modules has an associated transformed plan document. The identified set of software modules is invoked in the order specified in the decision tree. Each of the identified set of software modules performs operations using data from the transformed plan document associated with the software module. The identified set of software modules retrieves data from the computerized database and processes the retrieved data.

A combination of using digitized soil maps and location-determining technology can allow information to be gathered about characteristics of crops growing on the soil types in a field, then applying appropriate treatments at prescribed rates in response to the gathered characteristics. U.S. Pat. Nos. 6,160,902, issued 12 Dec. 2000 to Dickson et al., 6,178,253, issued 23 Jan. 2001 to Hendrickson et al., and 6,529,615, issued 4 Mar.2003 to Hendrickson et al., disclose a process for determining the health of crops in a field and for correcting deficiencies in the health of the crops. The process includes georeferencing aerial photographs of at least a portion of the field, the aerial photographs having a particular spatial resolution; determining the green plane in the aerial photographs; preparing a relative greenness map of the field based upon the nitrogen reference area, the relative greenness map providing crop status information having spatial resolution equivalent to the spatial resolution of the aerial photographs; converting the relative greenness map to a nitrogen recommendation map having spatial resolution equivalent to the spatial resolution of the photographs; and applying nitrogen to the field according to the nitrogen recommendation map, whereby the nitrogen is applied to the field without loss of spatial information. A process for treating crops is also disclosed. The process includes establishing, in a field to be treated, at least one predetermined area of high nitrogen reference; photographing from the air georeferenced portions of the field using a particular spatial resolution; differentiating soil and crops in the photographs thus obtained by segmenting images to select crop pixels; preparing a relative greenness map of the field from green plane based upon the high nitrogen reference area, the relative greenness map providing crop information having spatial resolution equivalent to the particular spatial resolution; and treating the crops in the field in accordance with the relative greenness map.

In another example of gathering information and using the information gathered to determine treatments to crop plants in a field, U.S. Pat. No. 6,549,852, issued 15 Apr. 2003 to Hanson, discloses methods and systems for characterizing and managing plots of land. Information related to elevation, soil conductivity, crop yield, and grower history is organized into profiles to generate a management zone profile. The management zone profile divides the plot of land into agronomy zones having attributable characteristics related to the elevation, soil conductivity, crop yield, and grower history information. The management zone profile is utilized to create a variable prescription of items, such as fertilizer, seed and pesticides, to be applied to the plot of land.

In yet another example of using digitized soil maps for gathering information and applying soil and crop treatments responsive to the gathered information, U.S. Pat. Nos. 6,199,000, issued 6 Mar. 2001, and 6,553,299, issued 22 Apr. 2003, both to Keller et al., disclose real time kinematic (RTK) global positioning system (GPS) technology integrated with precision farming methodologies to provide highly accurate seeding, cultivating, planting and/or harvesting operations. RTK GPS systems are used to control fully or semi-autonomous vehicles in these operations and may allow for precision planting of seeds (e.g., from a seeder equipped with an RTK GPS receiver and related equipment) and/or precision weed removal (e.g., using a vehicle fitted with weed eradication mechanisms such as augers and/or herbicide sprayers). Crop specific fertilizer/pesticide application is also enabled through the use of centimeter-level accurate positioning techniques.

In still yet another application for governing the rates and identities of soil and crop treatments, U.S. Patent Application Publication 2001/0002036, published 31 May 2001, and U.S. Pat. Nos. 5,979,703, issued 9 Nov. 1999, 6,000,577, issued 14 Dec. 1999, and 6,170,704, issued 9 Jan. 2001, all to Nystrom, disclose a mobile products applicator. The applicator includes a monitoring system particularly adaptable for use in selected product management applications, in which application rates for selected products are stored on-board one or more storage devices. The selected products are measured on the go and visually reported to an applicator operator in near real-time. The mobile products applicator provides environmental advantages to all through enhanced resource management by eliminating or significantly reducing ground and/or water contamination.

In yet another example of utilizing digital soil maps and location determining technology, U.S. Patent Application Publication 2002/0040300, published 4 Apr. 2002 and listing Ell as inventor, discloses a system and method for creating controller application maps for site-specific farming. The controller application maps can be used by an application machine to apply agricultural products to a field. The controller application maps are created by a mapping system by first accessing demo application maps. Demo application maps are broken into grids representing a field and containing a blend of agricultural products to apply to each cell of the grid or field. Demo application maps can be viewed or printed. The blend of agricultural products contained in demo application maps is in a Geographical Tagged Image File Format (GeoTIFF) containing unique data tags.

In still yet another example of using digitized soil maps, U.S. Patent Application Publication 2003/0036852, published 20 Feb. 2003 and listing Ell et al. as inventors, discloses a system and method for creating crop input requirement maps for site-specific farming. Crop input requirement maps contain a prescription of crop inputs for each section of a field. The prescription of crop inputs is used to create an application map. The first step in creating crop input requirement maps is to input recommendation equations into a mapping system. The user either selects a pre-defined recommendation equation or inputs an equation using mathematical equations, nested programming, or tables. Next, a field attribute map containing various agronomic data is accessed by the mapping system. The field attribute map includes data such as soil test values, elevation, desired crop yield, soil survey, as-applied data, yield monitor data, and other information. The final step combines the recommendation equations and field attribute maps to create a prescription of crop inputs for each section of a field.

In yet still another example, U.S. Patent Application Publication 2003/0208319, published 6 Nov. 2003 and listing Ell et al. as inventors, discloses system and method for creating demo application maps for site-specific farming. The demo application maps contain a blend of agricultural products. The blend of agricultural products can be viewed or printed either numerically or graphically. Once the demo application maps are combined with a map payment system, the blend of agricultural products can be used by an application machine to apply products to a field. The demo application maps are created by first inputting product information into a mapping system. The agricultural product information contains a percentage of crop inputs contained in the products. Next, the mapping system accesses crop input requirement maps. The crop input requirement maps contain a prescription of crop inputs to apply to a field. The product information and crop input requirement maps are combined to create a blend of agricultural products. The blend of agricultural products is then converted into a geographical tagged image file format with unique data tags.

The foregoing U.S. patents disclose utilizing digitized soil maps to precisely apply such materials as fertilizer components, seeding rates, and seeds of different varieties in response to the digitized soil maps by using global positioning systems and the like. However, none of these documents discloses or suggests using digitized soil maps and other data to select the most adapted crop varieties. Moreover, none of these documents discloses or suggests using digitized soil maps and other data to select and/or plant crop varieties most adapted to each agronomically significant soil type (or soil management category) within a field.

The present database includes and integrates characteristics of soil types and crop varieties. The soil types are those present in a geographical region, e.g., soil types present in fields in a county or in a specified region such as a sales area. The crop varieties are those adapted to the geographical region. An initial subset of the soil characteristics may be obtained from soil survey reports (e.g., from the USDA-Soil Conservation Service or from soil survey reports available in many counties) or from data gathered empirically. A final subset of the soil characteristics is derived from the characteristics obtained from the initial subset. However, the final subset can also be in part determined from commonly available sources pertaining to the soil survey reports, or, alternatively, empirical data obtained by tests conducted on soil samples from representative sites or from direct measurement of crop variety performance with respect to specific soil types. The final subset of soil characteristics includes indicators, e.g., indices, of the agronomic implications of one or more members of the initial subset. The final subset is used to group each of the soil types into agronomically important soil management categories. Crop varietal responses and recommendations to each of the soil management categories are then gathered from seed originators and, optionally, from other sources such as empirical data gathered by any of the foregoing protocols.

The soil management categories and crop varietal responses are then integrated such that each crop variety is recommended for one or more soil management category. The integration includes 1) indicating each category uniquely and graphically by one or more indicia such as a specific color and 2) indicating crop varieties adapted to each crop category with the same one or more indicia used to indicate the crop category.

While other means may be suitable, presenting the present soil management categories is contemplated to be done graphically by imposing these categories over digitized soil maps, e.g., using a computer to depict the unique one or more indicia, hence one or more management categories, on a screen. Crop varieties may be depicted in this manner as well, but may also be presented by a color-coded list printed on a sheet of paper. One example of the three subsets of soil characteristics is included in Table 1.

TABLE 1
Soil Characteristics of Harrison County, Iowa.

| Musym¹ | ///² | ER³ | Name⁴ | Profile⁵ | C yld⁶ | Sb yld⁷ | C⁸ | SB⁹ | cc¹⁰ | p¹¹ | N¹² | F¹³ | pHI¹⁴ | pH¹⁵ |
|---|---|---|---|---|---|---|---|---|---|---|---|---|---|---|
| 1C | C | 1 | Ida silt | silt loam | 124 | 42 | 3 | OD | 1 | 4 | | | 6.8 | 6.6-8.4 |
| | | | loam, 5 | to 60″ | | | | | | | | | | |
| | | | to 9 | | | | | | | | | | | |
| | | | percent | | | | | | | | | | | |
| | | | slopes | | | | | | | | | | | |
| 1C3 | C | 3 | Ida silt | silt loam | 111 | 37 | 2.1 | OD | 1 | 4 | | | 6.8 | 6.6-8.4 |
| | | | loam, 5 | to 60″ | | | | | | | | | | |
| | | | to 9 | | | | | | | | | | | |
| | | | percent | | | | | | | | | | | |
| | | | slopes, | | | | | | | | | | | |
| | | | severely | | | | | | | | | | | |
| | | | eroded | | | | | | | | | | | |
| 1D | D | 1 | Ida silt | silt loam | 115 | 39 | 2.1 | OD | 1 | 4 | | | 6.8 | 6.6-8.4 |
| | | | loam, 9 | to 60″ | | | | | | | | | | |
| | | | to 14 | | | | | | | | | | | |
| | | | percent | | | | | | | | | | | |
| | | | slopes | | | | | | | | | | | |
| 1D3 | D | 3 | Ida silt | silt loam | 102 | 34 | 2.1 | OD | 1 | 4 | | | 6.8 | 6.6-8.4 |
| | | | loam, 9 | to 60″ | | | | | | | | | | |
| | | | to 14 | | | | | | | | | | | |
| | | | percent | | | | | | | | | | | |
| | | | slopes, | | | | | | | | | | | |
| | | | severely | | | | | | | | | | | |
| | | | eroded | | | | | | | | | | | |
| 1E | E | 1 | Ida silt | silt loam | 98 | 33 | 2.1 | OD | 1 | 4 | | | 6.8 | 6.6-8.4 |
| | | | loam, 14 | to 60″ | | | | | | | | | | |
| | | | to 20 | | | | | | | | | | | |
| | | | percent | | | | | | | | | | | |
| | | | slopes | | | | | | | | | | | |
| 1E3 | E | 3 | Ida silt | silt loam | 85 | 28 | 2.1 | OD | 1 | 4 | | | 6.8 | 6.6-8.4 |
| | | | loam, 14 | to 60″ | | | | | | | | | | |
| | | | to 20 | | | | | | | | | | | |
| | | | percent | | | | | | | | | | | |
| | | | slopes, | | | | | | | | | | | |
| | | | severely | | | | | | | | | | | |
| | | | eroded | | | | | | | | | | | |
| 1F | F | 1 | Ida silt | silt loam | 0 | 0 | 2.1 | OD | 1 | 4 | | | 6.8 | 6.6-8.4 |
| | | | loam, 20 | to 60″ | | | | | | | | | | |
| | | | to 30 | | | | | | | | | | | |
| | | | percent | | | | | | | | | | | |
| | | | slopes | | | | | | | | | | | |
| 1F3 | F | 3 | Ida silt | silt loam | 0 | 0 | 2.1 | OD | 1 | 4 | | | 6.8 | 6.6-8.4 |
| | | | loam, 20 | to 60″ | | | | | | | | | | |
| | | | to 30 | | | | | | | | | | | |
| | | | percent | | | | | | | | | | | |
| | | | slopes, | | | | | | | | | | | |
| | | | severely | | | | | | | | | | | |
| | | | eroded | | | | | | | | | | | |
| 1G | G | 1 | Ida silt | silt loam | 0 | 0 | 2.1 | OD | 1 | 4 | | | 6.8 | 6.6-8.4 |
| | | | loam, 30 | to 60″ | | | | | | | | | | |
| | | | to 40 | | | | | | | | | | | |
| | | | percent | | | | | | | | | | | |
| | | | slopes | | | | | | | | | | | |
| 2G | G | 1 | Hamburg | coarse silt | 0 | 0 | 2.1 | O | ? | 4 | | | 7.8 | 7.4-8.4 |
| | | | silt loam, | loam to | | | | | | | | | | |
| | | | 40 to 75 | 65″ | | | | | | | | | | |
| | | | percent | | | | | | | | | | | |
| | | | slopes | | | | | | | | | | | |
| 3D | D | 1 | Castana | silt loam | 110 | 37 | 2.1 | OD | 1 | 4 | | | 7.8 | 7.4-8.4 |
| | | | silt loam, | to 60″ | | | | | | | | | | |
| | | | 5 to 14 | | | | | | | | | | | |
| | | | percent | | | | | | | | | | | |
| | | | slopes | | | | | | | | | | | |
| 3E | E | 1 | Castana | silt loam | 93 | 31 | 2.1 | OD | 1 | 4 | | | 7.8 | 7.4-8.4 |
| | | | silt loam, | to 60″ | | | | | | | | | | |
| | | | 14 to 20 | | | | | | | | | | | |
| | | | percent | | | | | | | | | | | |
| | | | slopes | | | | | | | | | | | |
| 10 | A | 1 | Monona | silt loam | 145 | 49 | 3 | O | 4 | 4 | | | 4.6 | 5.6-7.3 |
| | | | silt loam, | to 60″ | | | | | | | | | | |
| | | | 0 to 2 | | | | | | | | | | | |
| | | | percent | | | | | | | | | | | |
| | | | slopes | | | | | | | | | | | |
| 10B | B | 1 | Monona | silt loam | 142 | 48 | 3 | O | 4 | 4 | | | 4.6 | 5.6-7.3 |
| | | | silt loam, | to 60″ | | | | | | | | | | |
| | | | 2 to 5 | | | | | | | | | | | |
| | | | percent | | | | | | | | | | | |
| | | | slopes | | | | | | | | | | | |
| 10C | C | 1 | Monona | silt loam | 137 | 46 | 3 | O | 4 | 4 | | | 4.6 | 5.6-7.3 |
| | | | silt loam, | to 60″ | | | | | | | | | | |
| | | | 5 to 9 | | | | | | | | | | | |
| | | | percent | | | | | | | | | | | |
| | | | slopes | | | | | | | | | | | |
| 10C2 | C | 2 | Monona | silt loam | 133 | 45 | 2.1 | O | 4 | 4 | | | 4.6 | 5.6-7.3 |
| | | | silt loam, | to 60″ | | | | | | | | | | |
| | | | 5 to 9 | | | | | | | | | | | |
| | | | percent | | | | | | | | | | | |
| | | | slopes, | | | | | | | | | | | |
| | | | moderately | | | | | | | | | | | |
| | | | eroded | | | | | | | | | | | |
| 10D | D | 1 | Monona | silt loam | 128 | 43 | 2.1 | O | 4 | 4 | | | 4.6 | 5.6-7.3 |
| | | | silt loam, | to 60″ | | | | | | | | | | |
| | | | 9 to 14 | | | | | | | | | | | |
| | | | percent | | | | | | | | | | | |
| | | | slopes | | | | | | | | | | | |
| 10D2 | D | 2 | Monona | silt loam | 124 | 42 | 2.1 | O | 4 | 4 | | | 4.6 | 5.6-7.3 |
| | | | silt loam, | to 60″ | | | | | | | | | | |
| | | | 9 to 14 | | | | | | | | | | | |
| | | | percent | | | | | | | | | | | |
| | | | slopes, | | | | | | | | | | | |
| | | | moderately | | | | | | | | | | | |
| | | | eroded | | | | | | | | | | | |
| 10D3 | D | 3 | Monona | silt loam | 115 | 39 | 2.1 | O | 4 | 4 | | | 4.6 | 5.6-7.3 |
| | | | silt loam, | to 60″ | | | | | | | | | | |
| | | | 9 to 14 | | | | | | | | | | | |
| | | | percent | | | | | | | | | | | |
| | | | slopes, | | | | | | | | | | | |
| | | | severely | | | | | | | | | | | |
| | | | eroded | | | | | | | | | | | |
| 10E | E | 1 | Monona | silt loam | 111 | 37 | 2.1 | O | 4 | 4 | | | 4.6 | 5.6-7.3 |
| | | | silt loam, | to 60″ | | | | | | | | | | |
| | | | 14 to 20 | | | | | | | | | | | |
| | | | percent | | | | | | | | | | | |
| | | | slopes | | | | | | | | | | | |
| 10E2 | E | 2 | Monona | silt loam | 107 | 36 | 2.1 | O | 4 | 4 | | | 4.6 | 5.6-7.3 |
| | | | silt loam, | to 60″ | | | | | | | | | | |
| | | | 14 to 20 | | | | | | | | | | | |
| | | | percent | | | | | | | | | | | |
| | | | slopes, | | | | | | | | | | | |
| | | | moderately | | | | | | | | | | | |
| | | | eroded | | | | | | | | | | | |
| 10E3 | E | 3 | Monona | silt loam | 98 | 33 | 2.1 | O | 4 | 4 | | | 4.6 | 5.6-7.3 |
| | | | silt loam, | to 60″ | | | | | | | | | | |
| | | | 14 to 20 | | | | | | | | | | | |
| | | | percent | | | | | | | | | | | |
| | | | slopes, | | | | | | | | | | | |
| | | | severely | | | | | | | | | | | |
| | | | eroded | | | | | | | | | | | |
| 10F | F | 1 | Monona | silt loam | 0 | 0 | 2.1 | O | 4 | 4 | | | 4.6 | 5.6-7.3 |
| | | | silt loam, | to 60″ | | | | | | | | | | |
| | | | 20 to 30 | | | | | | | | | | | |
| | | | percent | | | | | | | | | | | |
| | | | slopes | | | | | | | | | | | |
| 10F2 | F | 2 | Monana | silt loam | 0 | 0 | 2.1 | O | 4 | 4 | | | 4.6 | 5.6-7.3 |
| | | | silt loam, | to 60″ | | | | | | | | | | |
| | | | 20 to 30 | | | | | | | | | | | |
| | | | percent | | | | | | | | | | | |
| | | | slopes, | | | | | | | | | | | |
| | | | moderately | | | | | | | | | | | |
| | | | eroded | | | | | | | | | | | |
| 10G | G | 1 | Monona | silt loam | 0 | 0 | 2.1 | O | 4 | 4 | | | 4.6 | 5.6-7.3 |
| | | | silt loam, | to 60″ | | | | | | | | | | |
| | | | 30 to 40 | | | | | | | | | | | |
| | | | percent | | | | | | | | | | | |
| | | | slopes | | | | | | | | | | | |
| 12B | B | 1 | Napier | silt loam | 130 | 44 | 3 | O | 4 | 4 | | | 5.6 | 6.1-7.3 |
| | | | silt loam, | to 60″ | | | | | | | | | | |
| | | | 2 to 5 | | | | | | | | | | | |
| | | | percent | | | | | | | | | | | |
| | | | slopes | | | | | | | | | | | |
| 12B+ | B | 1 | Napier | silt loam | 130 | 44 | 3 | O | 4 | 4 | | | 5.6 | 6.1-7.3 |
| | | | silt loam, | to 60″ | | | | | | | | | | |
| | | | 2 to 5 | | | | | | | | | | | |
| | | | percent | | | | | | | | | | | |
| | | | slopes, | | | | | | | | | | | |
| | | | overwash | | | | | | | | | | | |
| 12C | C | 1 | Napier | silt loam | 125 | 42 | 3 | O | 4 | 4 | | | 5.6 | 6.1-7.3 |
| | | | silt loam, | to 60″ | | | | | | | | | | |
| | | | 5 to 9 | | | | | | | | | | | |
| | | | percent | | | | | | | | | | | |
| | | | slopes | | | | | | | | | | | |
| 12D | D | 1 | Napier | silt loam | 116 | 39 | 2.1 | O | 4 | 4 | | | 5.6 | 6.1-7.3 |
| | | | silt loam, | to 60″ | | | | | | | | | | |
| | | | 9 to 14 | | | | | | | | | | | |
| | | | percent | | | | | | | | | | | |
| | | | slopes | | | | | | | | | | | |
| 17B | B | 1 | Napier- | Complex | 134 | 45 | 5 | D | 4 | 1 | | | 4.6 | 5.6-7.3 |
| | | | nodaway- | with 50 to | | | | | | | | | | |
| | | | colo | 60% | | | | | | | | | | |
| | | | complex, | Napier | | | | | | | | | | |
| | | | 2 to 5 | soil, 30 to | | | | | | | | | | |
| | | | percent | 40% | | | | | | | | | | |
| | | | slopes, | Nodaway | | | | | | | | | | |
| | | | occasional | soil, and | | | | | | | | | | |
| | | | brief | 10 to 20% | | | | | | | | | | |
| | | | flooding | Colo soil | | | | | | | | | | |
| 22D3 | D | 3 | Dow- | Complex | 97 | 32 | 2.1 | OD | 2 | 4 | | | 4.8 | 5.6-8.4 |
| | | | monona | with 60% | | | | | | | | | | |
| | | | silt | Dow soil | | | | | | | | | | |
| | | | loams, 9 | and 40% | | | | | | | | | | |
| | | | to 14 | Monona | | | | | | | | | | |
| | | | percent | soil | | | | | | | | | | |
| | | | slopes, | | | | | | | | | | | |
| | | | severely | | | | | | | | | | | |
| | | | eroded | | | | | | | | | | | |
| 22E3 | E | 3 | Dow- | Complex | 80 | 27 | 2.1 | OD | 2 | 4 | | | 4.8 | 5.6-8.4 |
| | | | monona | with 60% | | | | | | | | | | |
| | | | silt | Dow soil | | | | | | | | | | |
| | | | loams, 14 | and 40% | | | | | | | | | | |
| | | | to 20 | Monona | | | | | | | | | | |
| | | | percent | soil | | | | | | | | | | |
| | | | slopes, | | | | | | | | | | | |
| | | | severely | | | | | | | | | | | |
| | | | eroded | | | | | | | | | | | |
| 33D2 | D | 2 | Steinauer | clay loam | 102 | 0 | 2.1 | OD | ? | 3 | | | 7.8 | 7.4-8.4 |
| | | | clay | to 60″ | | | | | | | | | | |
| | | | loam, 9 | | | | | | | | | | | |
| | | | to 14 | | | | | | | | | | | |
| | | | percent | | | | | | | | | | | |
| | | | slopes, | | | | | | | | | | | |
| | | | moderately | | | | | | | | | | | |
| | | | eroded | | | | | | | | | | | |
| 733 | E | 3 | Steinauer | clay loam | 0 | 0 | 2.1 | OD | ? | 3 | | | 7.8 | 7.4-8.4 |
| E3 | | | clay | to 60″ | | | | | | | | | | |
| | | | loam, 14 | | | | | | | | | | | |
| | | | to 18 | | | | | | | | | | | |
| | | | percent | | | | | | | | | | | |
| | | | slopes, | | | | | | | | | | | |
| | | | severely | | | | | | | | | | | |
| | | | eroded | | | | | | | | | | | |
| 36 | A | 1 | Salix | silty clay | 145 | 49 | 3 | OD | 2 | 3 | | | 6.7 | 6.6-7.8 |
| | | | silty clay | loam to | | | | | | | | | | |
| | | | loam | 24″, silt | | | | | | | | | | |
| | | | | loam 24-60″ | | | | | | | | | | |
| 38 | A | 1 | Blake | Complex, | 128 | 43 | 5 | D | 1 | 1 | | | 6.8 | 6.6-8.4 |
| | | | and | the % s of | | | | | | | | | | |
| | | | Haynie | Blake and | | | | | | | | | | |
| | | | soils, | Haynie | | | | | | | | | | |
| | | | occasional | soils vary | | | | | | | | | | |
| | | | long | from one | | | | | | | | | | |
| | | | flooding | area to | | | | | | | | | | |
| | | | | another | | | | | | | | | | |
| 44 | A | 1 | Blencoe | silty clay | 120 | 40 | 5 | D | 4 | 1 | | | 5.6 | 6.1-7.3 |
| | | | silty clay | to 24″, | | | | | | | | | | |
| | | | | silty clay | | | | | | | | | | |
| | | | | loam 24-30″, | | | | | | | | | | |
| | | | | silt | | | | | | | | | | |
| | | | | loam 30-60″ | | | | | | | | | | |
| 46 | A | 1 | Keg silt | silt loam | 152 | 51 | 1 | O | 4 | 4 | | | 5.6 | 6.1-7.3 |
| | | | loam | to 60″ | | | | | | | | | | |
| 53 | A | 1 | Riverwash, | | 0 | 0 | 5 | D | ? | 1 | | | | ? |
| | | | long | | | | | | | | | | | |
| | | | ponding | | | | | | | | | | | |
| 66 | A | 1 | Luton | silty clay | 80 | 27 | 2.5 | D | 2 | 1 | | | 6.7 | 6.6-7.8 |
| | | | silty clay, | to 21″, | | | | | | | | | | |
| | | | occasional | silty clay | | | | | | | | | | |
| | | | brief | or clay | | | | | | | | | | |
| | | | flooding | 21-60″ | | | | | | | | | | |
| 66+ | A | 1 | Luton silt | silty clay | 100 | 34 | 5 | D | 2 | 1 | | | 6.7 | 6.6-7.8 |
| | | | loam, | to 21″, | | | | | | | | | | |
| | | | overwash, | silty clay | | | | | | | | | | |
| | | | occasional | or clay | | | | | | | | | | |
| | | | brief | 21-60″ | | | | | | | | | | |
| | | | flooding | | | | | | | | | | | |
| 67 | A | 1 | Woodbury | silty clay | 100 | 34 | 5 | D | 4 | 1 | | | 5.6 | 6.1-7.3 |
| | | | silty | to 24″, | | | | | | | | | | |
| | | | clay, | silty clay | | | | | | | | | | |
| | | | occasional | to silty | | | | | | | | | | |
| | | | brief | clay loam | | | | | | | | | | |
| | | | flooding | 24-35″, | | | | | | | | | | |
| | | | | silty clay | | | | | | | | | | |
| | | | | loam 35-60″ | | | | | | | | | | |
| 70 | A | 1 | Mcpaul | silt loam | 133 | 45 | 4 | D | 1 | 2 | | | 7.8 | 7.4-8.4 |
| | | | silt loam, | to 60″ | | | | | | | | | | |
| | | | occasional | | | | | | | | | | | |
| | | | very | | | | | | | | | | | |
| | | | brief | | | | | | | | | | | |
| | | | flooding | | | | | | | | | | | |
| 133 | A | 1 | Colo | silty clay | 140 | 47 | 5 | D | 4 | 1 | | | 4.6 | 5.6-7.3 |
| | | | silty clay | loam to | | | | | | | | | | |
| | | | loam, | 60″ | | | | | | | | | | |
| | | | occasional | | | | | | | | | | | |
| | | | long | | | | | | | | | | | |
| | | | flooding | | | | | | | | | | | |
| 133+ | A | 1 | Colo silt | silty clay | 135 | 45 | 5 | D | 4 | 1 | | | 4.6 | 5.6-7.3 |
| | | | loam, | loam to | | | | | | | | | | |
| | | | overwash, | 60″ | | | | | | | | | | |
| | | | occasional | | | | | | | | | | | |
| | | | long | | | | | | | | | | | |
| | | | flooding | | | | | | | | | | | |
| 137 | A | 1 | Haynie | silt loam | 126 | 42 | 4 | DO | 2 | 2 | | | 7.8 | 7.4-8.4 |
| | | | silt loam, | to 60″ | | | | | | | | | | |
| | | | occasional | | | | | | | | | | | |
| | | | very | | | | | | | | | | | |
| | | | brief | | | | | | | | | | | |
| | | | flooding | | | | | | | | | | | |
| 144 | A | 1 | Blake | silty clay | 130 | 44 | 4 | D | 1 | 2 | | | 7.8 | 7.4-8.4 |
| | | | silty clay | loam to | | | | | | | | | | |
| | | | loam, | 24″, silt | | | | | | | | | | |
| | | | rare brief | loam 24-60″ | | | | | | | | | | |
| | | | flooding | | | | | | | | | | | |
| 145 | A | 1 | Onawa | silty clay | 130 | 44 | 5 | D | 1 | 1 | | | 7.8 | 7.4-8.4 |
| | | | silt loam | to 6″, silty | | | | | | | | | | |
| | | | | clay or | | | | | | | | | | |
| | | | | clay 6-26″, | | | | | | | | | | |
| | | | | silt | | | | | | | | | | |
| | | | | loam 26-60″ | | | | | | | | | | |
| 146 | A | 1 | Onawa | silty clay | 120 | 40 | 5 | D | 1 | 1 | | | 7.8 | 7.4-8.4 |
| | | | silty clay | to 6″, silty | | | | | | | | | | |
| | | | | clay or | | | | | | | | | | |
| | | | | clay 6-26″, | | | | | | | | | | |
| | | | | silt | | | | | | | | | | |
| | | | | loam 26-60″ | | | | | | | | | | |
| 149 | A | 1 | Modale | silt loam | 126 | 42 | 5 | D | 1 | 1 | | | 7.8 | 7.4-8.4 |
| | | | silt loam, | to 22″, | | | | | | | | | | |
| | | | occasional | silty clay | | | | | | | | | | |
| | | | brief | 22-60″ | | | | | | | | | | |
| | | | flooding | | | | | | | | | | | |
| 156 | A | 1 | Albaton | silty clay | 100 | 34 | 2.5 | D | 1 | 1 | | | 7.8 | 7.4-8.4 |
| | | | silty clay | to 60″ | | | | | | | | | | |
| 157 | A | 1 | Albaton | silty clay | 105 | 35 | 2.5 | D | 1 | 1 | | | 7.8 | 7.4-8.4 |
| | | | silt loam, | to 60″ | | | | | | | | | | |
| | | | occasional | | | | | | | | | | | |
| | | | brief | | | | | | | | | | | |
| | | | flooding | | | | | | | | | | | |
| 212 | A | 1 | Kennebec | silt loam | 164 | 55 | 4 | DO | 4 | 2 | | | 4.6 | 5.6-7.3 |
| | | | silt | to 60″ | | | | | | | | | | |
| | | | loam, | | | | | | | | | | | |
| | | | occasional | | | | | | | | | | | |
| | | | brief | | | | | | | | | | | |
| | | | flooding | | | | | | | | | | | |
| 212+ | A | 1 | Kennebec | silt loam | 159 | 53 | 4 | DO | 4 | 2 | | | 4.6 | 5.6-7.3 |
| | | | silt | to 60″ | | | | | | | | | | |
| | | | loam, | | | | | | | | | | | |
| | | | overwash, | | | | | | | | | | | |
| | | | occasional | | | | | | | | | | | |
| | | | brief | | | | | | | | | | | |
| | | | flooding | | | | | | | | | | | |
| 220 | A | 1 | Nodaway | silt loam | 145 | 49 | 4 | DO | 4 | 2 | | | 5.6 | 6.1-7.3 |
| | | | silt loam, | to 60″ | | | | | | | | | | |
| | | | occasional | | | | | | | | | | | |
| | | | brief | | | | | | | | | | | |
| | | | flooding | | | | | | | | | | | |
| 237 | A | 1 | Sarpy | fine sand | 0 | 0 | 2.5 | D | 3 | 1 | | | 7.8 | 7.4-8.4 |
| | | | fine sand, | to 60″ | | | | | | | | | | |
| | | | 0 to 3 | | | | | | | | | | | |
| | | | percent | | | | | | | | | | | |
| | | | slopes, | | | | | | | | | | | |
| | | | occasional | | | | | | | | | | | |
| | | | long | | | | | | | | | | | |
| | | | flooding | | | | | | | | | | | |
| 237B | B | 1 | Sarpy | fine sand | 0 | 0 | 1 | DO | 1 | 4 | | | 7.8 | 7.4-8.4 |
| | | | fine sand, | to 60″ | | | | | | | | | | |
| | | | 3 to 7 | | | | | | | | | | | |
| | | | percent | | | | | | | | | | | |
| | | | slopes | | | | | | | | | | | |
| 238 | A | 1 | Sarpy | fine sand | 0 | 0 | 2.5 | D | 3 | 1 | | | 7.8 | 7.4-8.4 |
| | | | fine | to 60″ | | | | | | | | | | |
| | | | sandy | | | | | | | | | | | |
| | | | loam, 0 | | | | | | | | | | | |
| | | | to 3 | | | | | | | | | | | |
| | | | percent | | | | | | | | | | | |
| | | | slopes, | | | | | | | | | | | |
| | | | occasional | | | | | | | | | | | |
| | | | long | | | | | | | | | | | |
| | | | flooding | | | | | | | | | | | |
| 244 | A | 1 | Blend | silty clay | 108 | 36 | 2.5 | D | 4 | 1 | | | 4.6 | 5.6-7.3 |
| | | | silty clay | to 14″, | | | | | | | | | | |
| | | | | silty clay | | | | | | | | | | |
| | | | | loam 14-32″, | | | | | | | | | | |
| | | | | silty | | | | | | | | | | |
| | | | | clay 32-60″ | | | | | | | | | | |
| 255 | A | 1 | Cooper | silty clay | 126 | 42 | 5 | D | 2 | 1 | | | 5.6 | 6.1-7.3 |
| | | | silty clay | loam to | | | | | | | | | | |
| | | | loam | 26″, silty | | | | | | | | | | |
| | | | | clay 26-60″ | | | | | | | | | | |
| 275 | A | 1 | Moville | silt loam | 122 | 41 | 5 | D | 4 | 1 | | | 7.8 | 7.4-8.4 |
| | | | silt loam, | to 27″, | | | | | | | | | | |
| | | | occasional | silty clay | | | | | | | | | | |
| | | | brief | 27-33″, | | | | | | | | | | |
| | | | flooding | clay 33-60″ | | | | | | | | | | |
| 315 | A | 1 | Albaton | silty clay | 100 | 34 | 5 | D | 1 | 1 | | | 6.7 | 6.6-8.4 |
| | | | and sarpy | to 60″ | | | | | | | | | | |
| | | | soils, | | | | | | | | | | | |
| | | | frequent | | | | | | | | | | | |
| | | | long | | | | | | | | | | | |
| | | | flooding | | | | | | | | | | | |
| 436 | A | 1 | Lakeport | silty clay | 138 | 46 | 4 | DO | 4 | 2 | | | 5.6 | 6.1-7.3 |
| | | | silty clay | loam to | | | | | | | | | | |
| | | | loam | 35″, silty | | | | | | | | | | |
| | | | | clay 35-42″, silt | | | | | | | | | | |
| | | | | loam 42-60″ | | | | | | | | | | |
| 446 | A | 1 | Burcham | silt loam | 145 | 49 | 5 | D | 2 | 1 | | | 6.8 | 6.6-8.4 |
| | | | silt loam | to 26″, | | | | | | | | | | |
| | | | | silty clay | | | | | | | | | | |
| | | | | 26-60″ | | | | | | | | | | |
| 466 | A | 1 | Solomon | silty clay | 74 | 25 | 5 | D | 3 | 1 | | | 7.8 | 7.4-8.4 |
| | | | silty clay, | to 33″, | | | | | | | | | | |
| | | | frequent | clay 33-60″ | | | | | | | | | | |
| | | | long | | | | | | | | | | | |
| | | | flooding | | | | | | | | | | | |
| 514 | A | 1 | Grable | coarse silt | 103 | 35 | 2.5 | D | 1 | 2 | | | 7.8 | 7.4-8.4 |
| | | | silt loam, | loam to | | | | | | | | | | |
| | | | occasional | 23″, fine | | | | | | | | | | |
| | | | very | sand 23-60″ | | | | | | | | | | |
| | | | brief | | | | | | | | | | | |
| | | | flooding | | | | | | | | | | | |
| 515 | A | 1 | Percival | silty clay | 100 | 34 | 2.5 | DO | 2 | 2 | | | 7.8 | 7.4-8.4 |
| | | | silty clay, | to 24″, | | | | | | | | | | |
| | | | occasional | loamy | | | | | | | | | | |
| | | | very | fine sand | | | | | | | | | | |
| | | | brief | and fine | | | | | | | | | | |
| | | | flooding | sand 24-60″ | | | | | | | | | | |
| 516 | A | 1 | Vore | silty clay | 116 | 39 | 2.5 | D | 1 | 2 | | | 7.8 | 7.4-8.4 |
| | | | silty clay | loam to | | | | | | | | | | |
| | | | loam, | 24″, fine | | | | | | | | | | |
| | | | occasional | sand 24-60″ | | | | | | | | | | |
| | | | very | | | | | | | | | | | |
| | | | brief | | | | | | | | | | | |
| | | | flooding | | | | | | | | | | | |
| 538 | A | 1 | Carr very | very fine | 82 | 27 | 2.5 | DO | ? | 2 | | | 7.8 | 7.4-8.4 |
| | | | fine | sandy | | | | | | | | | | |
| | | | sandy | loam to | | | | | | | | | | |
| | | | loam, | 29″, | | | | | | | | | | |
| | | | occasional | loamy | | | | | | | | | | |
| | | | very | fine sand | | | | | | | | | | |
| | | | brief | and fine | | | | | | | | | | |
| | | | flooding | sand 29-60″ | | | | | | | | | | |
| 549 | A | 1 | Modale | silt loam | 106 | 36 | 5 | D | 1 | 1 | | | 7.8 | 7.4-8.4 |
| | | | very fine | to 22″, | | | | | | | | | | |
| | | | sandy | silty clay | | | | | | | | | | |
| | | | loam, | 22-60″ | | | | | | | | | | |
| | | | occasional | | | | | | | | | | | |
| | | | brief | | | | | | | | | | | |
| | | | flooding | | | | | | | | | | | |
| 550 | A | 1 | Borrow | | 0 | 0 | | | ? | | | | | ? |
| | | | pits | | | | | | | | | | | |
| 553 | A | 1 | Forney | silty clay | 91 | 30 | 2.5 | D | 2 | 1 | | | 5.7 | 6.1-7.8 |
| | | | silty clay, | to 8″, silty | | | | | | | | | | |
| | | | occasional | clay or | | | | | | | | | | |
| | | | brief | clay 8-19″, | | | | | | | | | | |
| | | | flooding | silty | | | | | | | | | | |
| | | | | clay 19-25″, | | | | | | | | | | |
| | | | | silty | | | | | | | | | | |
| | | | | clay or | | | | | | | | | | |
| | | | | clay 25-60″ | | | | | | | | | | |
| 717 C | C | 1 | Napier- | silt loam | 125 | 42 | 3 | O | 4 | 4 | | | 5.8 | 6.1-8.4 |
| | | | gullied | to 60″, | | | | | | | | | | |
| | | | land | half or | | | | | | | | | | |
| | | | complex, | more of | | | | | | | | | | |
| | | | 2 to 10 | the area is | | | | | | | | | | |
| | | | percent | gullied | | | | | | | | | | |
| | | | slopes, | | | | | | | | | | | |
| | | | typically | | | | | | | | | | | |
| | | | not | | | | | | | | | | | |
| | | | farmed | | | | | | | | | | | |
| 844 | A | 1 | Blake silt | silty clay | 132 | 44 | 4 | D | 1 | 2 | | | 7.8 | 7.4-8.4 |
| | | | loam, | loam to | | | | | | | | | | |
| | | | rare brief | 24″, | | | | | | | | | | |
| | | | flooding | coarse silt | | | | | | | | | | |
| | | | | loam 24-60″ | | | | | | | | | | |
| 849 | A | 1 | Kenmoor | fine sand | 90 | 30 | 2.5 | DO | ? | 1 | | | 6.8 | 6.6-8.4 |
| | | | fine sand, | to 25″, | | | | | | | | | | |
| | | | occasional | silty clay | | | | | | | | | | |
| | | | brief | 25-60″ | | | | | | | | | | |
| | | | flooding | | | | | | | | | | | |
| 866 | A | 1 | Luton | silty clay | 89 | 30 | 2.5 | D | 2 | 1 | | | 6 | 6.6-7.3 |
| | | | silty clay, | to 21″, | | | | | | | | | | |
| | | | thin | silty clay | | | | | | | | | | |
| | | | surface, | or clay | | | | | | | | | | |
| | | | occasional | 21-60″ | | | | | | | | | | |
| | | | brief | | | | | | | | | | | |
| | | | flooding | | | | | | | | | | | |
| T10 | A | 1 | Monona | silt loam | 145 | 49 | 3 | O | 4 | 4 | | | 4.6 | 5.6-7.3 |
| | | | silt loam, | to 60″ | | | | | | | | | | |
| | | | benches, | | | | | | | | | | | |
| | | | 0 to 2 | | | | | | | | | | | |
| | | | percent | | | | | | | | | | | |
| | | | slopes | | | | | | | | | | | |
| T10 B | B | 1 | Monona | silt loam | 142 | 48 | 3 | O | 4 | 4 | | | 4.6 | 5.6-7.3 |
| | | | silt loam, | to 60″ | | | | | | | | | | |
| | | | benches | | | | | | | | | | | |
| | | | 2 to 5 | | | | | | | | | | | |
| | | | percent | | | | | | | | | | | |
| | | | slopes | | | | | | | | | | | |

| | | | | | | | | Water | | | | |
|---|---|---|---|---|---|---|---|---|---|---|---|---|
| Musym¹ | H2O¹⁶ | Solum¹⁷ | PI¹⁸ | Permeability¹⁹ | RZ²⁰ | Tile²¹ | WTI²² | Table²³ | OMI²⁴ | OM²⁵ | CECI²⁶ | CEC²⁷ |
| 1C | 12.6 | | 4 | 0.6-2 | 5 | N | 4 | | 2.3 | 2.5-3.5 | 4 | 20-25 |
| 1C3 | 12.6 | | 4 | 0.6-2 | 5 | N | 4 | | 1.2 | 0.7-1.7 | 4 | 20-25 |
| 1D | 12.6 | | 4 | 0.6-2 | 5 | N | 4 | | 2.3 | 2.5-3.5 | 4 | 20-25 |
| 1D3 | 12.6 | | 4 | 0.6-2 | 5 | N | 4 | | 1.2 | 0.7-1.7 | 4 | 20-25 |
| 1E | 12.6 | | 4 | 0.6-2 | 5 | N | 4 | | 2.3 | 2.5-3.5 | 4 | 20-25 |
| 1E3 | 12.6 | | 4 | 0.6-2 | 5 | N | 4 | | 1.2 | 0.7-1.7 | 4 | 20-25 |
| 1F | 12.6 | | 4 | 0.6-2 | 5 | N | 4 | | 2.3 | 2.5-3.5 | 4 | 20-25 |
| 1F3 | 12.6 | | 4 | 0.6-2 | 5 | N | 4 | | 1.2 | 0.7-1.7 | 4 | 20-25 |
| 1G | 12.6 | | 4 | 0.6-2 | 5 | N | 4 | | 2.3 | 2.5-3.5 | 4 | 20-25 |
| 2G | 11.8 | | 4 | 0.6-2 | 6 | N | 4 | | 1.2 | 1.7-2.7 | 2.3 | 10-15 |
| 3D | 12.9 | | 4 | 0.6-2 | 5 | N | 4 | | 2.3 | 2.5-3.5 | 4 | 20-25 |
| 3E | 12.9 | | 4 | 0.6-2 | 5 | N | 4 | | 2.3 | 2.5-3.5 | 4 | 20-25 |
| 10 | 12.9 | | 4 | 0.6-2 | 5 | N | 4 | | 3 | 3.5-4.5 | 4.5 | 25-30 |
| 10B | 12.9 | | 4 | 0.6-2 | 5 | N | 4 | | 3 | 3.0-4.0 | 4.5 | 25-30 |
| 10C | 12.9 | | 4 | 0.6-2 | 5 | N | 4 | | 3 | 3.0-4.0 | 4.5 | 25-30 |
| 10C2 | 12.9 | | 4 | 0.6-2 | 5 | N | 4 | | 2.3 | 2.2-3.2 | 4.5 | 25-30 |
| 10D | 12.9 | | 4 | 0.6-2 | 5 | N | 4 | | 3 | 3.0-4.0 | 4.5 | 25-30 |
| 10D2 | 12.9 | | 4 | 0.6-2 | 5 | N | 4 | | 2.3 | 2.2-3.2 | 4.5 | 25-30 |
| 10D3 | 12.7 | | 4 | 0.6-2 | 5 | N | 4 | | 1.2 | 1.2-2.2 | 4.5 | 25-30 |
| 10E | 12.9 | | 4 | 0.6-2 | 5 | N | 4 | | 3 | 3.0-4.0 | 4.5 | 25-30 |
| 10E2 | 12.9 | | 4 | 0.6-2 | 5 | N | 4 | | 2.3 | 2.2-3.2 | 4.5 | 25-30 |
| 10E3 | 12.7 | | 4 | 0.6-2 | 5 | N | 4 | | 1.2 | 1.2-2.2 | 4.5 | 25-30 |
| 10F | 12.9 | | 4 | 0.6-2 | 5 | N | 4 | | 3 | 3.0-4.0 | 4.5 | 25-30 |
| 10F2 | 12.9 | | 4 | 0.6-2 | 5 | N | 4 | | 2.3 | 2.2-3.2 | 4.5 | 25-30 |
| 10G | 12.9 | | 4 | 0.6-2 | 5 | N | 4 | | 3 | 3.0-4.0 | 4.5 | 25-30 |
| 12B | 13.2 | | 4 | 0.6-2 | 5 | N | 4 | | 1.2 | 1.4-3.0 | 4 | 20-25 |
| 12B+ | 12.9 | | 4 | 0.6-2 | 5 | N | 4 | | 3 | 3.0-4.0 | 4 | 20-25 |
| 12C | 12.9 | | 4 | 0.6-2 | 5 | N | 4 | | 3 | 3.0-4.0 | 4 | 20-25 |
| 12D | 12.9 | | 4 | 0.6-2 | 5 | N | 4 | | 3 | 3.0-4.0 | 4 | 20-25 |
| 17B | | | 4 | 0.6-2 | 2 | 1 | 1 | 0-1 ft. | 1.4 | 1.0-6.0 | 4.6 | 20-41 |
| 22D3 | | | 4 | 0.6-2 | 5 | N | 4 | | 1.2 | 0.8-2.2 | 4.5 | 20-30 |
| 22E3 | | | 4 | 0.6-2 | 5 | N | 4 | | 1.2 | 0.8-2.2 | 4.5 | 20-30 |
| 33D2 | 9.9 | | 3 | 0.2-0.6 | 5 | N | 4 | | 2.3 | 2.2-3.2 | 4.5 | 25-30 |
| 733 | 9.9 | | 3 | 0.2-0.6 | 5 | N | 4 | | 1.2 | 1.2-2.2 | 4.5 | 25-30 |
| E3 | | | | | | | | | | | | |
| 36 | 12.5 | | 4 | 0.6-2 | 4 | 85 | 4 | 4-6 ft. | 3 | 3.0-4.0 | 5 | 30-36 |
| 38 | | | 4 | 0.6-2 | 3 | 1 | 4 | 2-4 ft. | 1.2 | 1.0-3.0 | 3.5 | 15-35 |
| 44 | 11.0 | | 2 | 0.06-2 | 2 | 85 | 3 | 1.5-3 ft. | 3.4 | 3.0-5.0 | 6 | 41-50 |
| 46 | 3.96 | | 4 | 0.6-2 | 4 | N | 4 | | 2.3 | 2.5-3.5 | 4.5 | 25-30 |
| 53 | | | | | | | 1 | 0 ft. | | | | |
| 66 | 7.6 | | 1 | <0.06-0.06 | 1 | N | 1 | 0-1 ft. | 3.4 | 3.0-5.0 | 6 | 41-200 |
| 66+ | 9.1 | | 1 | <0.06-0.06 | 1 | N | 1 | 0-1 ft. | 2 | 1.5-2.5 | 4 | 20-25 |
| 67 | 9.5 | | 2 | 0.06-2 | 2 | 75 | 1 | 0-1 ft. | 3.4 | 3.0-5.0 | 6 | 41-50 |
| 70 | 13.2 | | 4 | 0.6-2 | 4 | N | 4 | | 2 | 1.5-2.5 | 3 | 15-20 |
| 133 | 12.2 | | 4 | 0.6-2 | 2 | 72 | 1 | 0-1 ft. | 4.5 | 5.0-7.0 | 5.6 | 36-41 |
| 133+ | 12.0 | | 4 | 0.6-2 | 2 | 72 | 1 | 0-1 ft. | 3.4 | 3.0-5.0 | 4.5 | 25-30 |
| 137 | 12.3 | | 4 | 0.6-2 | 4 | N | 4 | 4-6 ft. | 1.2 | 1.0-3.0 | 3 | 15-20 |
| 144 | 12.6 | | 4 | 0.6-2 | 3 | 110 | 2 | 1-3.5 ft. | 1.2 | 1.4-3.0 | 4.5 | 25-35 |
| 145 | 11.1 | | 2 | 0.06-6 | 2 | N | 4 | 2-4 ft. | 1.2 | 1.0-2.0 | 3 | 15-20 |
| 146 | 11.1 | | 2 | 0.06-6 | 2 | N | 4 | 2-4 ft. | 2 | 2.0-3.0 | 5.6 | 36-41 |
| 149 | 9.4 | | 2 | 0.06-2 | 3 | N | 3 | 1.5-3 ft. | 1.2 | 1.4-3.0 | 3 | 15-20 |
| 156 | 7.2 | | 1 | <0.06-0.2 | 2 | N | 1 | 0-1 ft. | 1.2 | 1.4-3.0 | 5.6 | 36-41 |
| 157 | 8.1 | | 1 | <0.06-2 | 2 | N | 1 | 0-1 ft. | 2 | 1.5-2.5 | 4 | 20-25 |
| 212 | 13.4 | | 4 | 0.6-2 | 4 | N | 4 | 3-5 ft. | 3.4 | 4.0-6.0 | 5 | 30-36 |
| 212+ | 12.9 | | 4 | 0.6-2 | 4 | N | 4 | 3-5 ft. | 3.4 | 3.0-5.0 | 5 | 30-36 |
| 220 | 12.9 | | 4 | 0.6-2 | 4 | 75 | 4 | 4-6 ft. | 2 | 1.5-2.5 | 4 | 20-25 |
| 237 | 4.2 | | 6 | 6-20 | 7 | N | 4 | | 1 | 0.5-1.5 | 1.2 | 5.0-10 |
| 237B | 4.2 | | 6 | 6-20 | 7 | N | 4 | | 1 | 0.5-1.5 | 1.2 | 5.0-10 |
| 238 | 4.3 | | 5.6 | 2-20 | 7 | N | 4 | | 2 | 1.5-2.5 | 1.2 | 5.0-10 |
| 244 | 8.6 | | 1 | <0.06-2 | 2 | N | 1 | 0-1 ft. | 3.4 | 3.0-5.0 | 6 | 41-50 |
| 255 | 9.2 | | 2 | 0.06-2 | 3 | 35 | 4 | 2-5 ft. | 3 | 3.0-4.0 | 4.5 | 25-30 |
| 275 | 9.9 | | 1 | <0.06-2 | 3 | N | 2 | 1-3 ft. | 2 | 1.5-2.5 | 3 | 15-20 |
| 315 | | | 1 | <0.06-20 | | N | 1 | 0-1 ft. | 1.2 | 0.5-2.5 | 1.4 | 5.0-25 |
| 436 | 11.0 | | 3 | 0.1-2 | 3 | 85 | 4 | 2-4 ft. | 3 | 3.0-4.0 | 5 | 30-36 |
| 446 | 9.8 | | 2 | 0.06-2 | 4 | 45 | 4 | 2-5 ft. | 3 | 3.5-4.5 | 4 | 20-25 |
| 466 | 6.5 | | 1 | <0.06-0.06 | 1 | N | 1 | 0-1 ft. | 3.4 | 3.0-5.0 | 5.6 | 30-50 |
| 514 | 6.6 | | 4.7 | 0.6-20 | 6 | N | 4 | | 2 | 1.5-2.5 | 3 | 15-20 |
| 515 | 3.7 | | 2 | 0.06-20 | 3 | N | 4 | 2-4 ft. | 1.2 | 1.0-3.0 | 5.6 | 36-41 |
| 516 | 7.1 | | 4.7 | 0.6-20 | 4 | N | 4 | 2-4 ft. | 2 | 2.0-3.0 | 4.5 | 25-30 |
| 538 | 6.9 | | 5.6 | 2-20 | 7 | N | 4 | | 1 | 0.5-1.5 | 3 | 15-20 |
| 549 | 9.4 | | 2 | 0.06-2 | 3 | N | 3 | 1.5-3 ft. | 1.2 | 1.0-2.0 | 3 | 15-20 |
| 550 | | | | | | | | | | | | |
| 553 | 7.2 | | 1 | <0.06-0.06 | 2 | N | 1 | 0-1 ft. | 2.3 | 2.4-4.0 | 5.6 | 36-41 |
| 717 C | | | 4 | 0.6-2 | 5 | N | 4 | | 3 | 3.0-4.0 | 4 | 20-25 |
| 844 | 12.6 | | 4 | 0.6-2 | 3 | 110 | 2 | 1-3.5 ft. | 2 | 1.5-2.5 | 4 | 20-25 |
| 849 | 7.7 | | 2 | 0.06-20 | 4 | N | 4 | 2.5-3 ft. | 1 | 0.5-1.5 | 2.3 | 10-15 |
| 866 | 7.6 | | 1 | <0.06-0.06 | 1 | N | 1 | 0-1 ft. | 3.4 | 3.0-5.0 | 6 | 41-41 |
| T10 | 12.9 | | 4 | 0.6-2 | 5 | N | 4 | | 3 | 3.0-4.0 | 4.5 | 25-30 |
| T10 B | 12.9 | | 4 | 0.6-2 | 5 | N | 4 | | 3 | 3.0-4.0 | 4.5 | 25-30 |

¹Musym. Alphanumeric classification encompassing the soil type and other agronomically relevant characteristic(s).
²///. Slope Index.
³ER. Eroded soil index.
⁴Name. Soil series name.
⁵Profile. Soil texture(s) present.
⁶C yld. Typical corn yield (bushels per acre).
⁷Sb yld. Typical soybean yield (bushels per acre).
⁸C. Corn seed category.
⁹SB. Soybean seed category.
¹⁰cc. Calcium carbonate index.
¹¹p. Phytophthora/fungi potential index.
¹²N. Nitrogen management index.
¹³F. Nutrient Zone Management.
¹⁴pHI. pH index.
¹⁵pH. Soil pH.
¹⁶H2O. H2O index.
¹⁷Solum. Total soil depth supporting biological activity.
¹⁸PI. Soil Permeability index.
¹⁹Permeability.
²⁰RZ. Root zone drainage index.
²¹Tile. Tile line interval (feet).
²²WTI. High water table index (water table index).
²³Water Table.
²⁴OMI. Organic matter index.
²⁵OM. Organic matter content (%).
²⁶CECI. Cation exchange capacity index.
²⁷CEC. Cation exchange capacity.

I. Initial subset.

The first subset of the exemplary soil characteristics is obtained from sources such as soil survey reports and related materials, from further analysis of representative soil samples, and/or from empirical data gathered during farming. One way of gathering additional data during farming is including a history of crop productivity data such as grain yields and/or grain moistures at harvest from sectors obtained by the protocol disclosed in U.S. Pat. No. 6,029,106. From the above soil characteristics, the initial subset includes:

A. Slope. Slope is measured in units rise (fall) per hundred horizontal units and is expressed as a percentage. Exemplary slope classifications include:
0 to 2% slopes, nearly level;
2 to 6% slopes, gently sloping;
6 to 12% slopes, sloping;
12 to 18% slopes, moderately steep, often not farmed;
18 to 25% slopes, steep, typically not farmed; and
25 to 45% slopes, very steep, typically not farmed.

B. Name. Name of the soil type obtained from, e.g., soil survey reports.

C. Profile. The profile is the soil texture or combination of soil textures typically present in a 60-inch profile.

D. Typical corn yield. Typical corn yield is the corn yield (bu/ac) normally obtained from the soil type with the other enumerated characteristics and is obtainable from county office records, e.g., Farm Services Agency county offices. Alternatively, Typical Corn Yield can be determined empirically by recording yields from a typical soil type over a number of growing seasons.

E. Typical soybean yield. Typical soybean yield is the soybean yield (bu/ac) normally obtained from the soil type with the other enumerated characteristics and obtainable from county office records, e.g., Farm Services Agency county offices. Alternatively, typical soybean yield can be determined empirically by recording yields from a typical soil type over a number of growing seasons.

F. pH. Soil pH is the pH of the soil solution. pH, in turn, is defined as the negative logarithm of the hydrogen ion (H+) concentration. In the context of the instant invention, pH is the soil pH typically encountered from the soil type in the region, given the other enumerated characteristics. Alternatively, pH of the soil solution can be determined empirically for a specific soil type.

G. Solum. A solum is an upper set of horizons (e.g., A, E, B) present in a soil that related through the same cycle of pedogenic (soil forming) processes and considered to be the portion of the soil capable of supporting and sustaining economic crop growth and development.

H. Permeability. Permeability is the ease with which gases, liquids, or plant roots penetrate or pass through a bulk mass of soil or a layer of soil. In the context of the present invention, permeability is measured as the rate (inches/hour) at which water infiltrates the soil type in the region, given the other enumerated characteristics.

I. Tile. Tile line intervals are noted in feet separating tile runs. Presently the instant tile line maps are limited to a single standard, ⅜-inch drainage coefficient at a three feet depth. This standard was chosen because it is commonly used in published recommendations by several states. However, future versions may include coefficients and depths published by states using any standard. Additionally, the instant category may include tile line intervals and depths as determined by the “Ellipse Equation” versus tested data for states without published data sets. The Ellipse Equation may be used where soil saturation is the result of a high water table with a restrictive soil layer and the hydrology has been (or will be) altered with drains (surface or subsurface). The Ellipse Equation calculates the steady state drawdown condition for a given flow rate. The flow rate is expressed as a depth of water removed per unit of time (inches/day) which is called a drainage coefficient. The Ellipse Equation assumes that rainfall is occurring at the same time as drainage is occurring. Drainage coefficients used in the Ellipse Equation should be based on the site climate and soil water storage capacity. A more complete description can be found in US Department of Agriculture, Natural Resources Conservation Service, 1997, Hydrology Tools for Wetland Determination, Chapter 19, Engineering Field Handbook. Donald E. Woodward (ed.). USDA, NRCS, Fort Worth, Tex. In the Ellipse Equation, S, the parallel drain spacing, is calculated:
S=\sqrt{(4K)(m²+2am)/q}
where,
S=parallel drain spacing (ft);
K=weighted hydraulic conductivity above the restrictive layer (in/hr);
m=vertical distance after drawdown, of water table above drain and at midpoint between drains (ft);
a=depth of barrier (impermeable layer) below drain (ft); and
q=drainage rate (in/hr).

The variable “m” can be calculated from other parameters, where:
m=d-c, in which,
d=depth to drain from ground (or reference elevation) surface (ft); and
c=depth to water table from ground (or reference elevation) surface after the evaluation period (ft).

Exemplary tile line intervals include:
0. None needed;
1. A “complex” of soils; refer to the recommended tile line interval for individual soils in the complex; and
11111. No published data.

J. Organic matter. Soil organic matter expressed as percentages. Soil organic matter, in turn, is the aggregate term referring to the organic constituents in the soil, including undecayed plant and animal tissues, their partial decomposition products, and the soil biomass. Soil organic matter is frequently said to consist of humic substances and nonhumic substances. Nonhumic substances can be placed in one of the categories of discrete compounds such as sugars, amino acids, and fats. Humic substances are the other, unidentifiable components. The organic matter percentages reported are those typically encountered for the soil type with other enumerated characteristics and can be obtained from, e.g., the soil survey reports or related records. Alternatively, soil organic matter percentages can be determined empirically.

K. Cation exchange capacity. Cation exchange capacity (CEC) is the sum of exchangeable bases plus total soil acidity at a specific pH, value, usually 7.0 or 8.0. CEC is usually expressed as centimoles of charge per kilogram of exchanger (cmol_{c}kg⁻¹) or millimoles of charge per kilogram of exchanger, but may also be expressed as milliequivalents per 100 grams of soil. Cation exchange capacities depicted herein may be those typically encountered for a given soil type with other enumerated characteristics and can be obtained from the soil survey reports, related records, or empirically, e.g., by measuring CEC of representative samples of a particular soil type in a specific field.

II. Final Subset.

From the above soil characteristics, the final subset includes:

A. Slope index. Slope Index is derived from the slopes typically present and includes:
A. 0 to 2% slopes, nearly level;
B. 2 to 6% slopes, gently sloping;
C. 6 to 12% slopes, sloping; an
D. 12 to 18% slopes, moderately steep, often not farmed;
E. 18 to 25% slopes, steep, typically not farmed; and
F. 25 to 45% slopes, very steep, typically not farmed.

B. Eroded soil index. Eroded Soil Index measures the degree of soil erosion at a specific site.
1. No erosion indicated in data (lack of color (clear) are used on maps);
2. Eroded soil; and
3. Severely eroded soil.

C. Water Table Index. Water Table Index measures the seasonal high water table as the highest water level of a specific saturated, undrained soil. The first number in the range given is the determinant for this index.
1. Plow layer and above (½ foot below to 3 feet above the soil surface).
   +3-1 foot;
   +2-1 foot (e.g., Oldham and Quam Silty Clay Loams);
   +1-1 foot (e.g., Lura, Glencoe, Blue Earth, Talcot);
   0-1½ foot;
   0-2 feet (e.g., Millington and Comfrey);
   0-3 feet;
   ½-1 foot;
   ½-1½]foot;
   ½-2 feet; and
   ½-3 feet.
2. Below plow layer (1 foot below soil surface, subject to 10 ton axle load compaction) Disease potential (fungi) is present if compacted.
   1-2 feet (e.g., Delft and Jeffers Clay Loams);
   1-2½ feet; and
   1-3 feet (e.g., Webster Clay Loam).
3. Below plow layer (1½ below soil surface, subject to 20 ton axle load compaction) Disease potential (fungi) is present if compacted.
   1½-3 feet (e.g., Jeffers Variant Clay Loam).
4. Non-problematic (2 feet to deeper than 6 feet below soil surface).
   2-3½ feet (e.g., Guckeen Silty Clay Loam);
   2-4 feet (e.g., Crippin Clay Loam);
   2-5 feet (e.g., Collinwood Silty Clay);
   2½-5 feet (e.g., Nicollet Loam);
   2½-6 feet;
   3½-6 feet;
   4-6 feet;
   >6 feet.

D. Soil Permeability Index. The Soil Permeability Index is an indication of the ability of a specific soil type to allow water to move through the soil profile. Permeability is measured as the inches per hour water moves through a saturated soil. Typical terms describing soil permeability are:

| | | |
|---|---|---|
| Very slow | <0.06 | inch |
| Slow | 0.06-0.2 | inch |
| Moderately slow | 0.2-0.6 | inch |
| Moderate | 0.6-2.0 | inches |
| Moderately rapid | 2.0-6.0 | inches |
| Rapid | 6.0-20.0 | inches |
| Very rapid | >20 | inches |

As an intermediate step, soil permeabilities are further characterized as:
1. Very slow
   <0.06 inch.
2. Slow.
   0.06-0.2 inch (e.g., Lura Silty Clay)
   0.06-0.6 inch (e.g., Fulda Silty Clay)
   0.06-2.0 inches
   0.06-6.0 inches
3. Moderately slow.
   0.2-0.6 inch (e.g., Waldorf, Collinwood, Ransom, Rushmore, Glencoe)
   0.2-2.0 inches (e.g., Webster Clay Loam)
   0.2-6.0 inches
   0.2-20.0 inches
4. Moderate.
   0.6-2.0 inches (e.g., Clarion, Nicollet Loams)
   0.6-6.0 inches (e.g., Linder Loam)
   0.6-20.0 inches (Biscay, Mayer, Wadena, Fairhaven)
5. Moderately rapid.
   2.0-6.0 inches (e.g., Dickman, Estherville)
   2.0-20.0 inches (e.g., Dickinson Loam)
6. Rapid.
   6.0-20.0 inches (e.g., Sioux Sandy Loam)
   >6.0 inches
7. Very Rapid.
   >20.0 inches

Soil Permeability Index. (throughout 5-foot profile). From the above characteristics and characterization, the following Soil Permeability Indices are assigned by using the most restrictive (slowest) permeability within a five-foot profile or to a root restrictive layer.
1. Very slow permeable layer in the soil profile, less than 0.06 inch per hour.
2. Slow permeable layer in the soil profile, 0.06 inch per hour series.
3. Moderately slow permeable layer in the soil profile, 0.2 inch/hour series.
4. Moderate (ideal) permeability, 0.6 inch per hour series.
5. Moderately rapid permeability, 2 to 6 inches per hour.
6. Rapid permeability, 6 to 20 inches per hour series.
7. Very rapid permeability, greater than 20 inches per hour.

E. Natural Root Zone Drainage Index. As an intermediate step, the Natural Root Zone Drainage classifications of the soil types are characterized as being:
1. Very Poorly Drained. Water percolates through the soil so slowly that free water remains at, or on, the soil surface most of the growing season. Unless the soil is artificially drained, most mesophytic crops cannot be grown. Very poorly drained soils are commonly level or depressed and are frequently ponded.
2. Poorly Drained. Water percolates through the soil so slowly that the soil remains saturated periodically during the growing season or for long periods of time. Free water is commonly at, or near, the soil surface for sufficient time during the growing season that most mesophytic crops cannot be grown unless the soil is artificially drained. The soil is not continuously saturated in layers directly below plow depth. Poor drainage results from a high water table, a slowly pervious layer within the profile, seepage, nearly continuous rainfall, or a combination thereof (e.g., Webster Clay Loam).
3. Somewhat Poorly Drained. Water percolates to the soil sufficiently slowly that the soil is saturated for significant periods during the growing season. Soil saturation during these significant periods markedly restricts the growth of mesophytic crops unless artificial drainage is provided. Somewhat poorly drained soils commonly have a slowly pervious layer, a high water table, additional water from seepage, nearly continuous rainfall, or a combination thereof (e.g., Crippen Loam).
4. Moderately Well Drained. Water percolates through the soil somewhat slowly during some periods of the growing season. Moderately well drained soils are wet for only a short period of time during the growing season, but periodically are sufficiently saturated that most mesophytic crops are adversely affected. Moderately well drained soils commonly have a slowly pervious layer within, or directly below, the solum, or periodically receive high rainfall, or both (e.g., Nicollet Loam).
5. Well Drained. Water percolates through the soil readily, but not rapidly. Water is available to crop plants throughout most of the growing season and wetness does not inhibit root growth for significant periods during most growing seasons. Well drained soils are commonly medium textured and are mainly free from mottling (e.g., Clarion Loam).
6. Somewhat Excessively Drained. Water percolates from the soil rapidly. Many somewhat excessively drained soils are sandy and rapidly pervious. Some are shallow. Moreover, some are so steep that much of the precipitation received is lost as runoff.
7. Excessively Drained. Water percolates from the soil very rapidly. Excessively drained soils are commonly very coarse textured, rocky, or shallow. Some excessively drained soils have steep slopes.

Rooting Zone Drainage Index (throughout 5-foot profile). From the foregoing, the instant Rooting Zone Drainage Indices are assigned.
1. Very Poorly Drained, D soybeans & corn category 5.
2. Poorly Drained, D/O soybean & corn category 4.
3. Somewhat Poorly Drained, D/O soybeans & corn category 4.
4. Moderately Well Drained, O/D soybeans & corn category 3.
5. Well Drained, Offensive soybeans & corn category 3.
6. Somewhat Excessively Well Drained, Offensive soybeans & corn category 1.
7. Excessively Drained, Offensive soybeans & corn category 1.

F. Soil Texture Index. Soil texture is the relative proportion of sand, silt, and clay particles in a mass of soil. The present soil texture Index includes 12 recognized basic textural classes in order of increasing proportions of fine particles. These basic classes can then be subdivided by specifying coarse,” “fine,” or “very fine.” Some soil survey descriptions also include non-textural terms, such as “muck” and “peat.”

Coarse Textured Soils

| | |
|---|---|
| 0.75 | Coarse Sand |
| 1.0 | Sand |
| 1.25 | Fine Sand |
| 1.5 | Very Fine Sand |
| 1.75 | Loamy Coarse Sand |
| 2.0 | Loamy Sand |
| 2.25 | Loamy Fine Sand, Mucky Loamy Sand |
| 2.5 | Loamy Very Fine Sand, Mucky Loamy Fine Sand |

Moderately Coarse Textured Soils

| | |
|---|---|
| 2.75 | Coarse Sandy Loam |
| 3.0 | Sandy Loam (e.g., Dickman, Dickinson, Estherville) |
| 3.25 | Fine Sandy Loam (e.g., Grogan) |
| 3.5 | Very Fine Sandy Loam |
| 4.0 | Loam (e.g., Clarion and Nicollet Loams) |
| 4.5 | Peat |
| 4.75 | Mucky Peat |
| 5.0 | Silt Loam (e.g., Truman) |
| 5.25 | Mucky Silt Loam - (35 - Blue Earth) |
| 5.5 | Muck (e.g., Palms and 154 - Blue Earth) |
| 6.0 | Silt |
| 7.0 | Sandy Clay Loam |

Moderately Fine Textured Soils

| | |
|---|---|
| 8.0 | Clay Loam (E.g., Webster Clay Loam) |
| 9.0 | Silty Clay Loam (e.g., Spicer) |

Fine Textured Soils

| | |
|---|---|
| 10.0 | Sandy Clay |
| 11.0 | Silty Clay (e.g., Collinwood, Lura, Waldorf) |
| 12.0 | Clay |

G. Available Water Capacity Index. The present Available Water Capacity Index measures the relative capacity of soils to retain water for use by crop plants. Water holding capacity of a soil is typically defined as the difference between the amount of soil water at field capacity and the amount of soil water at the wilting point of crop plants, and is most commonly expressed as inches of water per foot of soil. The present Available Water Capacity Index expresses the water retaining capacity of soils as inches per 60-inch profile, or as inches to a limiting (impervious) layer.

| | | |
|---|---|---|
| 1. | Very Low | 0-3 inches; |
| 2. | Low | 3-6 inches (e.g., Estherville, Dickman, Linder); |
| 3. | Moderate | 6-9 inches (e.g., Mayer, Dickinson, Wadena, Fairhaven, Collinwood); |
| 4. | High | 9-12 inches (e.g., Nicollet, Clarion); and |
| 5. | Very High | >12 inches (e.g., Webster, Lakefield, Delft, Primghar). |

H. Soil Reaction Index—pHI. The present Soil Reaction Index is a measure of the acidity or alkalinity of the soil.

| | | |
|---|---|---|
| 1. | Extremely Acid | <4.5; |
| 2. | Very Strongly Acid | 4.5-5.0; |
| 3. | Strongly Acid | 5.1-5.5 (e.g., Rolfe Silt Loam); |
| 4. | Medium Acid | 5.6-6.0 (e.g., Fairhaven, Sac, Grogan, Dickinson, Everly); |
| 5. | Slightly Acid | 6.1-6.5 (e.g., Nicollet, Clarion, Waldorf, Kingston, Collinwood); |
| 6. | Neutral | 6.6-7.3 (e.g., Webster, Glencoe, Comfrey, Wilmonton, Lura, Letri); |
| 7. | Mildly Alkaline | 7.4-7.8 (Jeffers, Swanlake, Crippen, Delft, Mayer, Canisteo, Storden); |
| 8. | Moderately Alkaline | 7.9-8.4 (e.g., Lakefield, Millington, Vallers, Knoke, Buse); |
| 9. | Strongly Alkaline | 8.5-9.0; and |
| 10. | Very Strongly Alkaline | 9.1 and higher. |

I. Soluble Salts

| | |
|---|---|
| Very Low | 0.01-0.25 millihos per centimeter; |
| Low | 0.26-0.50 millihos per centimeter; |
| Medium | 0.51-0.75 millihos per centimeter; |
| High | 0.76-2.0 millihos per centimeter; and |
| Very High | 2.1 millihos per centimeter and higher. |

J. Calcium Carbonate Equivalent (CCE)

| | |
|---|---|
| Low | 0-2.5%; |
| Medium | 2.6-5.0%; and |
| High | >5.0%. |

K. Iron Chlorosis Severity Index. Using measures of Calcium Carbonate Equivalent (CCE) and soluble salts, an indicator termed “risk” is determined.

| CCE | Salt Level | Risk* |
|---|---|---|
| 0-2.5% | <0.5 mmhos/cm | low; |
| 0-2.5% | >0.5-1.0 mmhos/cm | moderate; |
| 0-2.5% | >1.0 mmhos/cm | high; |
| 2.6-5.0% | 0-0.25 mmhos/cm | low; |
| 2.6-5.0% | 0.26 - mmhos/cm | moderate; |
| 2.6-5.0% | 0.51-1.0 mmhos/cm | high; |
| 2.6-5.0% | >1.0 mmhos/cm | very high; |
| >5.0% | 0-0.25 mmhos/cm | moderate; |
| >5.0% | 0.26-0.50 mmhos/cm | high; |
| >5.0% | 0.51-1.0 mmhos/cm | very high; and |
| >5.0% | >1.0 mmhos/cm | extreme. |

*Low - iron chlorosis in soybeans is not likely in fields with these characteristics based on CCE and salt levels.
*Moderate - iron chlorosis in soybeans may develop in some areas of these fields under wet cool conditions, based on CCE and salt levels. Planting a variety tolerant to iron chlorosis is advised.
*High - Iron chlorosis is likely to develop in some areas of fields with these characteristics under wet cool conditions based on CCE and salt levels. Planting a variety tolerant to iron chlorosis is advised.
*Very High - Iron chlorosis may be severe in these fields under wet cool conditions based on CCE and salt levels. Planting a variety tolerant to iron chlorosis is strongly advised.
*Extreme - Iron chlorosis will be severe in these fields under wet cool conditions based on CCE and salt levels. Severe iron chlorosis may severely reduce yield. Soybeans are not recommended in these fields.

Calcium Carbonate (CaCO₃) Index (Top Layer of the Soil Profile, with Exception Noted in Index 3).

1. High Calcium carbonate (5% or greater in the first number of the range given, e.g., 5 -10, 5-15, 5-30);

1.1. High Calcium carbonate on Sloping Soils (“C” slopes and greater);

2. Probably high Calcium carbonate (greater than 5% in the second number of the range given if not already designated as Index 1, e.g., 0-10, 0-15, 1-15);

2.1 Probable High Calcium carbonate on Sloping Soils (“C” slopes and greater);

3. Possible high Calcium carbonate (5% or less in the second number of a range, e.g., 0-3, 0-5, 1-3, 1-5; an important inclusion is the example of MN 662 Nora soil type in which the first layer is 0, but the second layer is 8 inches or less from the soil surface and has a Calcium carbonate potential that could be mixed with the plow layer through tillage);

4. Calcium carbonate not probable (0% Calcium carbonate in the first layer);

6. Typically not farmed; and

7. Data not estimated.

Phytophthora/Fungi (Potential) Index.

1. Protect against Phytophthora, seed treatment recommended;

2. Increased risk of Phytophthora, seed treatment suggested;

3. Phytophthora is possible;

4. Low probability of Phytophthora; and

6. Typically Not Farmed.

Organic Matter Index: (Topsoil).

1. Very Low, 0.1 to 1.5%;

2. Low, 1.6 to 3.0%;

3. Medium, 3.1 to 4.5%;

4. High, 4.6 to 6.0%; and

5. Very High, over 6.1%.

Cation Exchange Capacity Index, (Topsoil Data—If Available).

1. Sand, 0 to 8;

2. Loamy Sand, 9 to 12;

3. Silty Loam/Sandy Loam, 13 to 20;

4. Loam, 21 to 28;

5. Clay Loam, 29 to 40; and

6. Clay/Peat, over 40.

C. Crop Placement Categories. Utilizing the foregoing information, Seed Placement Categories are then determined, e.g., for corn and soybeans. The present Seed Placement Categories are described as:

Corn Seed Placement Categories.

1. Drought Probable. Includes soils with Plant Available Water Capacity Indices of 1 (very low, 0 to 3 inches) and 2 (low, 3 to 6 inches); and typically contains sand/gravel, within a five-foot profile.

1.1. Drought Probable. Includes soils with bedrock/cemented stone, or other root restrictive zone within a 5 foot profile with 6 inches or less of plant available water.

2. Drought Possible. Includes soils with a Plant Available Water Capacity Index of 3 (moderate, 6 to 9 inches); typically contains sand/gravel within a five-foot profile; and also includes the following sub-categories.

2.1. Drought Possible/Eroded Slopes. Includes eroded soil with slope indices of C, D, E, and F. These soils have shallow and eroded topsoil that may have limited nutrients needed for efficient use of available water. Also, eroded soils are prone to runoff because they typically have less organic matter needed to hold water and repair soil structure for adequate water infiltration.

2.2. Drought Possible. Includes soils with bedrock/cemented stone or other root restrictive zone within a 5 foot profile with moderate (6-9 inches) plant available water.

2.3. Drought Possible/High Water Table/Eroded Slopes. Includes eroded soils with high water tables, which also seep. This sub-category includes the soils in sub-category 2.1. The water properties of these soils require a seed selection for cool/wet soils early in the growing season and a seed selection that can tolerate drought later in the growing season, when the seeping ceases.

2.5. Drought Possible/High Water Table. High Water Table Sands: These soils typically begin a growing season saturated with water and end the growing season with insufficient water for good crop development and growth (droughty). This category requires hybrids with a combination of good early cool and wet characteristics and good drought tolerance. This category is defined as a combination of a Plant Available Water Capacity Index of 3 or less (less than 9 inches of plant available water) and Seasonal High Water Table Indices 1, 2 or 3 (e.g., a water table above plow layer or within a foot of the soil surface; hence subject to compaction by a ten-ton axle load or a water table 1½ feet below the soil surface and subject to 20 ton axle load compaction); or “Perched,” “Ponded,” or “Flooded” water features; or Permeability Indices of 1 and 2 (<0.06 and 0.06 series).

3. Non-problematic Soils. This category contains soils with the following characteristics: 1) a non-problematic rooting zone drainage, e.g., Index 4 (moderately well drained) and better; 2) no permeability problems, e.g., Index 3 (moderately slow, 0.2 inches per hour series) and better; and 3) no seasonal high water table or non-problematic seasonal high water table, e.g., Index 4 (two feet below soil surface and deeper series).

4. Possibly Poorly Drained. Seed treatment suggested. This category contains either of the following characteristics: Rooting Zone Drainage Indices 2 or 3 (poorly drained and somewhat poorly drained), and Seasonal High Water Table Indices 2 and 3 (one foot below surface and therefore subject to compaction by ten-ton axel loads, also 1½ foot below soil surface and therefore subject to compaction by twenty-ton axle loads), or Occasional Very Brief Flooding from streams or adjacent slopes.

5. Probably Poorly Drained. Seed treatment is recommended. This category contains soils with any one of the following characteristics: 1) Soil Texture Index of 12 (clay), a Rooting Zone Drainage Index of 1 (very poorly drained), Soil Permeability Indices of 1 or 2 (very slow and slow, 0.06 inch per hour and slower series), Seasonal High Water Table Index of 1 (water ranges from above soil surface to plow layer depth), “Frequent flooding,” “ponded” or “perched water” are usually in the soil description.

6. Typically Not Farmed.

Soybean Seed Placement Categories.

Soybean Placement Categories for Phytophthora and/or Other Fungi.

Offensive. No Phytophthora predisposition because of all of the following characteristics: Soil Texture Indices 1 to 5 (sand, loamy sand, sandy loam, loam, and silt loam); Non-problematic Seasonal High Water Table, Seasonal High Water Table Index 4 (two feet and greater); Rooting Zone Drainage Index 5 (well-drained), and better; Permeability Index 4 (moderate, 0.6 inches water per hour series) and better.

Offensive/Defensive. Phytophthora and other fungal diseases are possible because of one or more of the following characteristics: Soil Texture Indices 6 through 11 (silt, sandy clay loam, clay loam, silty clay loam, sandy clay, and silty clay)—these are finer soils that both wick and hold water; Seasonal High Water Table Index 3 (1½ feet deep), therefore susceptible to compaction by twenty-ton axel load that would increase wicking of water in the soil; Rooting Zone Drainage Index 4 (moderately well drained); Permeability Index 3 (moderately slow, 0.2 inch water per hour series); rare, very brief flooding.

Defensive/Offensive. This category contains one or more of the following characteristics: Seasonal High Water Table Index 2 (one foot series and therefore subject to compaction from ten-ton axle load that would increase wicking of water in the soil); Occasional very brief flooding from streams or adjacent slopes; Rooting Zone Drainage Indices 2 and 3 (somewhat poorly and poorly drained).

Defensive. This category contains any of the following characteristics: Soil Texture Index 12 (clay); Seasonal High Water Table Index 1 (water table ranges from above soil surface to plow layer; Frequent Very Brief Flooding; Rooting Zone Drainage Index 1 (very poorly drained); Permeability Indices 1 and 2 (very slow to slow, 0.06 inches per hour series); either “Ponded” or “Perched” as a soil water feature. Soybean Placement Categories for Iron Chlorosis Deficiency (Chlorosis. Soil Complexes are assigned a soybean variety placement category that has the most problematic characteristic of any soils in the complex.

Offensive (O). Up to a 7.2 pH, an indicator that neither soluble salts nor Calcium carbonate (CCE) poses a chlorosis problem. Using CCE as a criterion, CCE 0-2.5% with <0.5 mmhos/cm or CCE 2.6-5% with <.25 mmhos/cm. These soils are considered as non-problematic with a low chlorosis potential. These soils are not likely to experience chlorosis.

Offensive/Defensive (O/D). pH 7.3 to 7.5 is one indicator. However, 0-2.5% CCE with >0.5-1.0 mmhos/cm or 2.6-5% CCE with 0.26-0.50 mmhos/cm or >5% CCE with 0-0.25 mmhos/cm are also believed to impart moderate chlorosis potential. These soils may develop chlorosis under cool wet conditions; automatically includes glacial till “C” slopes or greater with 5% or greater CCE.

Defensive/Offensive (D/O). pH 7.6 to 7.7 is one indicator. Alternatively 0-2.5% CCE with >1.0 mmhos/cm or 2.6-5% CCE with 0.51-1.0 mmhos/cm or >5% CCE with 0.26-0.50 mmhos/cm may also be indicative of this classification. These soils are likely to develop chlorosis under cool wet conditions. Hence, seed treatment is suggested.

Defensive (D). pH 7.8 and above is one indicator. Alternatively, 2.6-5% CCE with >1.0 mmhos/cm may be an indicator of a very high risk for chlorosis; >5% CCE with >0.51 mmhos/cm may indicate very high to an extreme risk of chlorosis. Thus, iron chlorosis may be severe in this field under cool wet conditions and seed treatment is recommended.

Typically not farmed.

Crop Varietal Characteristics. Information about crop varieties to be characterized is obtained from originators, e.g., seed companies.

Corn Hybrids. One exemplary set of corn characteristics for each variety includes:
1. Relative maturity;
2. Characterization as to seed/soil categories within defined soil characteristics;
3. Whether the hybrid tolerates low soil fertility (N, P, K, and Zn) and suggested population
ranges in low fertility soils.
4. Whether the hybrid tolerates salts resulting from poorly drained soils.
5. Whether the hybrid is adapted to narrow rows (e.g., 15, 18, 20, or 22 inch row widths) and suggested population ranges.
6. Whether the hybrid is adapted to no-till management and suggested population ranges with respect to each of the soil categories (e.g., sand to wet clay).
7. The suggested ridge-till placement with respect to different soil categories (e.g., sand to wet clay).
8. Whether the hybrid is adapted to manured situations (e.g., from swine feeding and rearing operations).
9. Whether the hybrid has a “massive” or “penetrating” root system, or both.
10. Whether the ear is flex, semi-flex, or fixed.
11. Whether the hybrid has glyphosate tolerance, Liberty® and/or Lightning® tolerance, insect tolerance/resistance, and the sources of these tolerances.
12. The genetics of the hybrid.
13. Whether the hybrid has increased value for ethanol and/or corn syrup production.
14. Whether the hybrid has increased nitrogen efficiency, drought tolerance, cold tolerance, and/or enhanced nutrient levels for human and/or livestock nutrition.

Soybean Varieties. After the soil type characteristics are determined, the characteristics for each crop variety are obtained, e.g., from the seed company (originator) marketing the variety or from empirical data obtained from observation and experimentation. The seed originator classifies the soybean variety in one or more of the management categories and further classifies the soybean variety for reaction to soybean cyst nematode, white mold, and BSR. The soybean cultivar is yet further classified for suitability to no-till culture. Two other major concerns are addressed: 1) iron chlorosis potential and 2) Phytophthora/fungi potential.

Chlorosis: rating of 1-4.
1. Very high chlorosis potential (2.6-5% CCE with >1.0 mmhos/cm salts or >5% CCE with 0.51-1.0 mmhos/cm salts). Iron chlorosis may be severe in fields with these characteristics under cool wet conditions. Examples are Canisteo (54) clay loam, Spicer (45) silty clay loam, Talcot (36), and Mayer (37) loams. The seed company is asked to list soybean varieties which can tolerate soils with these conditions, listing best to worst.
2. High chlorosis potential (0-2.5% CCE with >1.0 mmhos/cm salts, or 2.6%-5% CCE with 0.51-1.0 mmhos/cm salts, or >5% CCE with 0.26-0.5 mmhos/cm salts). Soils with these characteristics are likely to develop chlorosis under cool wet conditions. Exemplary soils with these characteristics include Crippen (52) & Delft (52) clay loams, Biscay (44) & Linder (39) loams. The seed company is asked to list soybean varieties which can tolerate high chlorosis conditions, listing best to worst.
3. Moderate chlorosis potential (0-2.5% CCE with 0.6-1.0 mmhos/cm salts, or 2.6-5% CCE with 0.26-0.5 mmhos/cm salts, or >5% CCE with 0-0.25 mmhos/cm salts). Soils with these traits may develop chlorosis under cool wet conditions. Glencoe (48) clay loam, Oldham (47) silt clay loam are exemplary soils possessing these characteristics. The seed company is asked to list varieties tolerating moderate chlorosis from best to worst.
4. Non-problematic—Low chlorosis potential (0-2.5% CCE with <0.5 mmhos/cm salts, or 2.6-5.0% CCE with 0-0.25 mmhos/cm salts). Examples of non-problematic soils include Nicollet (54) and Clarion (54) loams. These soils are not likely to experience chlorosis. The seed company is asked to list the varieties that are adapted to non-problematic soils from best to worst.
   Phytophthora/Soil Fungi Potential

The instant invention categorizes Phytophthora/soil fungi potential based on the presence of excess soil water (zones of water saturation)—the height of the soil water in the soil, how tightly the soil water is held by the soil, how quickly the soil water moves thru the soil.

Information is requested as to how the seed originator places/balances both specific resistance (i.e. Rps1-a thru k) and Field Tolerance (Resistance) rating in the following categories.
1.—Soils being usually wet in the spring and requiring little rain spell to become saturated during the growing season. Blue Earth (39) mucky silt loam, Glencoe (48) clay loam, Lura (46) silty clay, are examples. The seed originator is asked to list varieties that are best in this situation, best to worst in this category—and to annotate each variety's specific resistance (if any) and/or field tolerance.
2.—Soils subject to compaction, raising the water table. The seasonal high water table is one foot below the soil surface, poor drainage, and occasional brief flooding from streams or adjacent slopes. Webster (52) and Canisteo (54) clay loams, Waldorf (50) silty clay, are examples. The originator is asked to list varieties classified here, best to worst in this category, with annotations on specific resistance and/or tolerance.
3.—Soils in this soybean category are the best corn yielding ground, but can have occasional Phytophthora problems in soybeans. The soil textures in this category are finer textures which wick and hold water, can suffer from heavy implement compaction, and may not have the best rooting drainage. Nicollet (54) loam, Collinwood (47) silty clay, Everly (52) clay loam, Galva (51) silty clay loam, are examples. The seed originator is asked to list the beans placed here, with annotations on specific resistance, field tolerance, or both, best to worst.
4.—Soils in this category range from sands to some silt clays, but all share the same qualities—well-drained surfaces and rooting zones. There are no Phytophthora predispositions associated with these soils unless an extreme year occurs. Clarion (54) loam, Fairhaven (38) silt loam, Estherville (21) sandy loam, are examples. The seed originator is asked to list soybean varieties in this category with the last soybean variety being the one with the least tolerance to Phytophthora - and to annotate them with the originator's rating(s).

Other questions characterizing soybean cultivars include:
1. To indicate any soybean varieties that can tolerate droughty conditions better than others in normal tillage? The term “drought tolerant” is seldom used in the description of a soybean variety.
2. To comment on managing and list varieties for no-till or reduced till droughty soil conditions. Some farmers with sandy soils generally no-till drill or skip-row their soybeans. Hence, these farmers need residue on the soil surface and an early canopy to reduce evaporation. The seed originator is asked whether a tall soybean cultivar is recommended for these conditions.
3. Manure on soybeans. Soybean yields are known to increase with manure applications. However, it is suggested farmers not exceed 2000 gallons of pit manure to prevent damage from salts. Also, manure is an ideal culture medium for fungi. Thus, soybeans with high disease tolerance are suggested when manuring is practices. The seed originator is asked to comment on these statements and recommend soybean cultivars for these conditions.

### EXAMPLE 1

First Protocol for Determining Seed Placement Categories.

Corn:

1. Soils determined to have 6 inches and less plant available water from column H2O are assigned to Corn Placement Category 1, Drought Probable.

2. All soils identified as “Frequently Flooded” are annotated as such in the Name column. This annotation is used to assign that soil to Corn Placement Category 5, Probably Poorly Drained.

3. All soils identified as “Ponded” are annotated as such in the Name column. That annotation is used to designate that soil to Corn Placement Category 5, Probably Poorly Drained.

4. All soils identified as “perched” are annotated as such in the Name column. That annotation is used to assign that soil to Corn Placement Category 5, Probably Poorly Drained.

5. All soils designated as “clay” texture in the top (first) profile in the Profile column are assigned to Corn Placement Category, Probably Poorly Drained.

6. From the present Natural Root Drainage Index, all soils designated “1” in the RZ column are assigned to Corn Placement Category 5, probably poorly drained.

7. From the present Soil Permeability Index, all soils designated as either a “1” or “2” in the PI column are assigned to Corn Placement Category 5, Probably Poorly Drained.

8. From the Slope column (designated//), all soils labeled “C” which are also labeled “2” or “3” from the Erosion column (designated Er) and that are also labeled “1” or “2” from the Seasonal High Water Table Index column (designated WTI) are assigned Corn Placement Category 2.3, Drought Possible, Steep Soils That Seep.

9. C2 or C3 soils remaining after step 8 above, plus all D, E, and F Slope Indices are assigned Corn Placement Category 2.1, drought possible, Eroded C and all D, E, and F soil.

10. From H2O column, all soils with nine inches and less of Plant Available Water, that also have a Seasonal High Water Table Index (column WTI) of a “1” are assigned Corn Placement Category 2.5, Drought Possible, High Water Table Soils.

11. All soils identified as “Occasionally Flooded” are identified as such in the Name column. That annotation is used to assign that particular soil to Corn Placement Category 4, Possibly Poorly Drained.

12. From Rooting Zone Drainage Index column (labeled RZ), all soils annotated “2” or “3”, also annotated “2” or “3” in the Seasonal High Water Table Index column (labeled WTI) are assigned to Corn Hybrid Seed Placement Category 4, Possibly Poorly Drained.

13. All soils with Plant Available Water Capacity (column H2O) from 6.1 inches to 9 inches are assigned to Corn Hybrid Seed Placement Category 2, Drought Possible.

14. All remaining soils are assigned Corn Hybrid Seed Placement Category 3, Non-Problematic.

Soybeans.

Chlorosis.

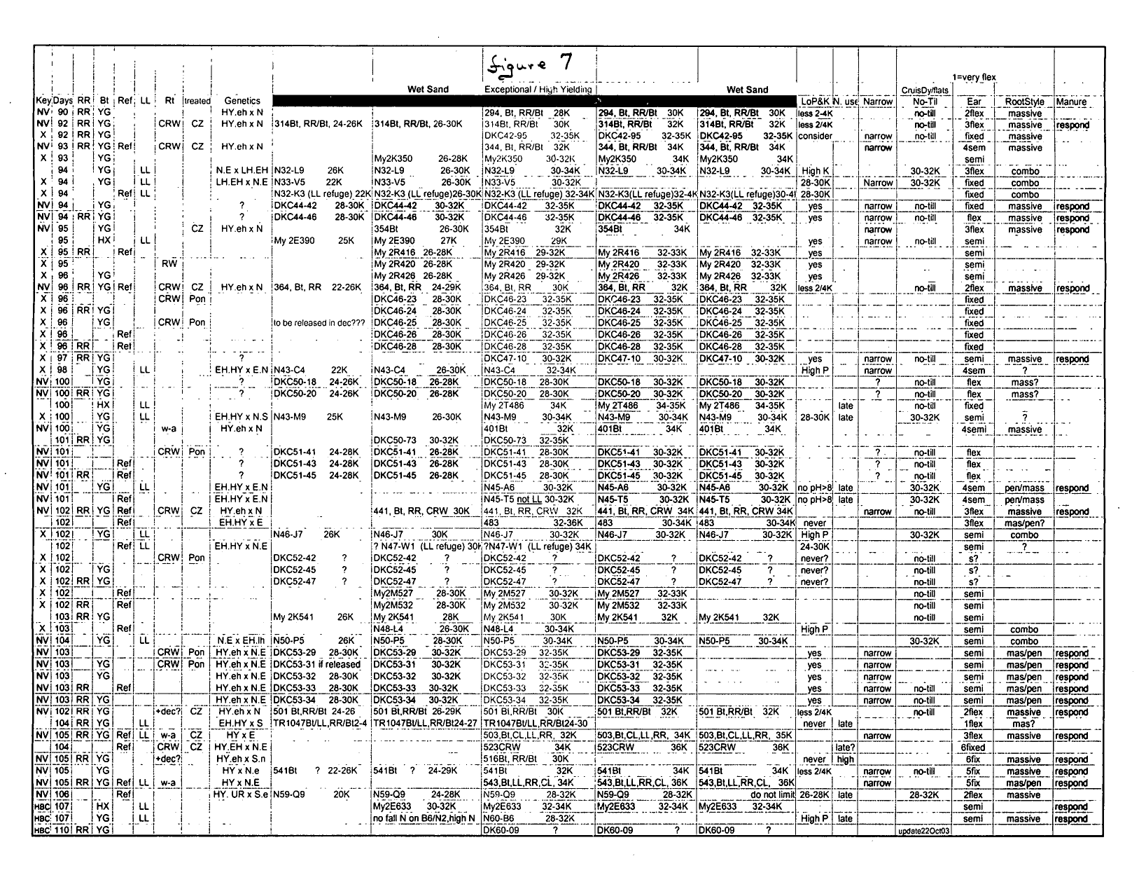

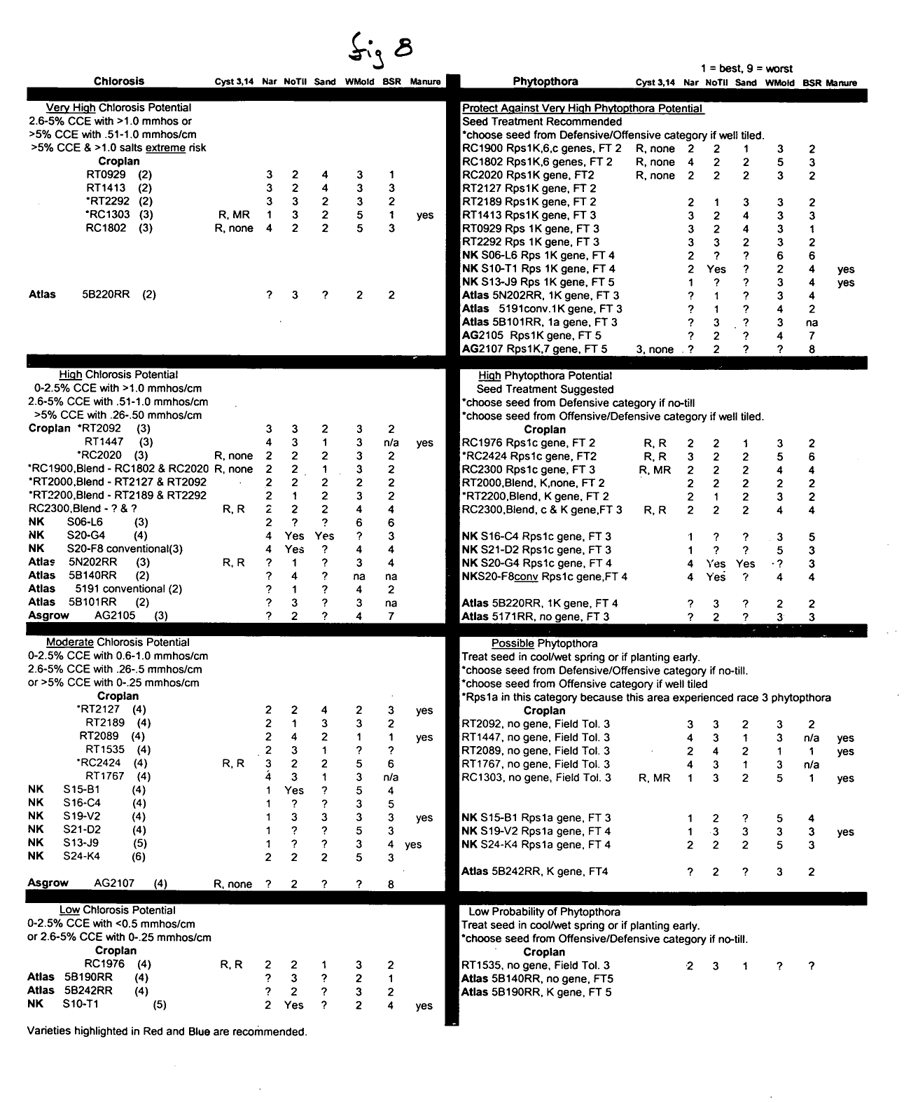

1. Soils identified as already qualifying (5% minimum in the range) for a “High CCE” designation (Index 1), that also are labeled as having “C” or greater slopes are assigned a 2.1 in the Calcium Carbonate Equivalent column (labeled cc). Commercially available soybean varieties suggested for this subcategory are listed in the exemplary Soybean Variety Seed Placement Chlorosis Category under the “Offensive/Defensive” placement; e.g., see left one-half of the page, FIG. 8.

2. Soils identified in 1 (Index 1) above, that are not labeled as “C” slopes or greater are assigned a “1” in the Calcium Carbonate Equivalent column (labeled cc). Commercially available soybean varieties suggested for this subcategory are listed in the Soybean Variety Seed Placement Chlorosis Category under the “Defensive” placement; e.g., see left one-half of FIG. 8.

3. Soils identified as having a Calcium Carbonate Equivalent (CCE) range exceeding 5% (high Calcium carbonate) in the upper part of the range, are assigned to Index 2 and a “2” is assigned to that soil in the Calcium Carbonate Equivalent column (labeled cc). Commercially available soybean varieties suggested for this subcategory are listed in the Soybean Variety Seed Placement Chlorosis Category under the “Defensive/Offensive” placement; see, e.g., left one-half of FIG. 8.

4. Soils identified as having a Calcium Carbonate Equivalent (CCE) range probably not exceeding 5% (high Calcium carbonate) are assigned to Index 3 and a “3” is assigned to that soil in the Calcium Carbonate Equivalent column (labeled cc). Commercially available soybean varieties suggested for this subcategory are listed in the Soybean Variety Placement Chlorosis Category under the “Offensive/Defensive” placement; see e.g., left one-half of FIG. 8.

5. Soils identified as having ” 0%” Calcium Carbonate Equivalent are assigned to Index 4 and a “4” is assigned to that soil in the Calcium Carbonate Equivalent column (labeled cc). All commercially available soybean varieties can be raised in this subcategory labeled as “Offensive.”

Phytophthora Potential.

1. From the present Soil Permeability Index (PI), all soils designated with either “1” or “2” in the PI column are assigned a “1” in the Phytophthora column (labeled p). It is noted that Ranges are identified because they are not whole numbers, but often include a decimal point. If a decimal point is encountered, refer to the first digit of the number and ignore the decimal point and all numbers following the decimal point.

2. From the present Rooting Zone Drainage Index (RZ), all soils designated as a “1” in the RZ spreadsheet column are assigned a “1” and the Phytophthora column (labeled p).

3. All soils identified as “Frequently Flooded” are annotated as such in the Name column. This annotation is used to assign designated soils with a “1” in the Phytophthora column (labeled p).

4. From the present High Water Table Index (WTI), all soils designated with “1” in the WTI column are assigned a “1” in the Phytophthora column (labeled p).

5. All soils identified as clay soils in the Name column are assigned a “1” in the Phytophthora column (labeled p).

After the foregoing soils are in excluded from further consideration,

6. From the present Rooting Zone Drainage Index (RZ), all soils designated with either “2” or “3” and the RZ column are assigned a “2” and the Phytophthora column (labeled p).

7. All soils identified as “Occasionally Flooded” are identified as such in the Name column. This annotation is used to assign these soils with a “2” in the Phytophthora column (labeled p).

8. From the present High Water Table Index, all soils designated with a “2” in the WTI column are assigned a “2” in the Phytophthora column (labeled p).

After excluding all soils designated supra,

9. From the present Permeability Index (PI), all soils designated with a “3” in the PI column are assigned a “3” in the Phytophthora column (labeled p).

10. From the present High Water Table Index (WTI), all soils designated with a “3” in the WTI column, are assigned a “3” in the Phytophthora column (labeled p).

11. From the present Rooting Zone Drainage Index (RZ), all soils designated with a “4” in the RZ column, are assigned a “3” in the Phytophthora column (labeled p).

12. All soils identified as silt, sandy clay loam, clay loam, silty clay loam, sandy clay, or silty clay in the Name column are assigned a “3” in the Phytophthora column (labeled p).

13. All soils remaining from the foregoing steps are assigned a “4” in the Phytophthora column (labeled p).

In assigning an overall Soybean Category designation (e.g., Defensive, Defensive/Offensive, Offensive/Defensive, or Offensive) the numbers in the “p” and “cc” columns are compared. The lowest number in each of the foregoing columns is selected. If the lowest number is a 1, the particular soil type is designated as a “Defensive” seed/soil category. If the lowest number is a “2”, the seed/soil category is designated as “Defensive/Offense.” If the lowest number is a “3,” the seed/soil category is designated as “Offensive/Defensive.” If both columns have a numeral 4, the seed/soil category is designated as “Offensive.”

Seed/Soil Management Categories with accompanying color-coded characteristic visuals are highly adaptable and further uses are expected. For example, an adaptation might be used for a single farmer to implement a planting population map for the computer on this farmer's corn planter. The soils on the farmer's farm could be categorized using the present Seed/Soil Management Categories and planting populations were varied as the farmer planted the fields in the “Zones” created. Another adaptation is in creating “Zones” for soil testing using the “Nutrient Zone Management” category. Other uses include recommendations and suggestions for such cultural practices as seed treatments; manure application husbandry; commercial fertilizer husbandry; pollution potential from fertilizers, insecticides, and herbicides; tillage practices; erosion control; adaptable crops; irrigation adaptability; and irrigation management.

The present Seed/Soil Management Categories are insightful and unique, especially when used in conjunction with accompanying color-coded indicia.

The present Seed/Soil Management Categories comprise unique: soil categories, seed categories, management categories, seed management categories, soil management categories, and management recommendation categories because they are defined and determined by an insightful and unique combination of soil and water characteristics.

The present Seed/Soil Management Categories with integrated color-coded visuals are insightful and unique and are of great value to the farmer and seed originator for reducing risk and maximizing yields and income.

Nutrient Management. In addition to selecting crop varieties, the present seed placement categories also provide insights on managing fertilizer amendments such as nitrogen, phosphorous, potassium, zinc, sulfur, and lime (pH adjustment).

Nitrogen Management Index.

1. (corn category 1) Leaching is probable because of sand/gravel in the soil profile. Do not fall apply nitrogen. Consider split applications of N.

2. (corn category 2) Leaching is possible because of sand/gravel in the soil profile. Fall applied nitrogen is probably not recommended.

3. (corn categories 1.1, 2.2, 2.3, and 2.5) Both denitrification and leaching are possible, do not fall apply nitrogen. Some of these soils may contain gravel, bedrock or other root restrictive zone within a five-foot profile. Consider split-shot or spoon-feeding nitrogen.

4. (corn categories 2.1 and 3) Non-problematic for nitrogen management. Follow best management practices for your area.

5. (corn category 4) Denitrification is possible due to poor internal soil drainage. Consider split applications of N. Be ready to side-dress nitrogen.

6. (corn category 5) Denitrification is possible due to poor internal soil drainage. Fall applications of N are probably not recommended. Follow best management practices for your area. Consider split applications of N. Be ready to side-dress nitrogen.

7. Typically not farmed.

Nutrient Zone Management.

1. (corn category 1) Porous/droughty soils. K and S concerns. Insure that K and S are sufficient. If this is either an isolated area or one of a few isolated areas in a field, P and K fertility soil tests may be higher that the remainder of the field. Fertilizer applications may have exceeded crop removals. Use extreme caution with “pop-up ” (on the seed) starter fertilizers when planting corn. If this porous/droughty soil is dry at seeding depths, do not apply pop-up fertilizer because desiccation of either the seed or seedling roots is possible.

2. (corn category 1 and CaCO₃ 1, 1.1, 2, 2.1, and 3) Porous/droughty, Calcium carbonate soils. P, K, S, and Zn concerns. Insure K and S levels are sufficient. Potentially high levels of Calcium carbonate may affect the availability of P and Zn. Use extreme caution with “pop-up” (on the seed) starter fertilizers when planting corn. If this porous/droughty soil is dry at seeding depth, do not apply any pop-up fertilizer. Desiccation of either the seed or seedling roots is possible.

3. (corn category 1.1, 2, and 2.2) Possible droughty soils. Insure K is sufficient for efficient use of limited soil water. If this is an isolated area or one of a few isolated areas in a field, P and K fertility test levels may be higher than the rest of the field. Fertilizer applications may have exceeded crop removals.

4. (corn categories 1.1, 2, and 2.2, with CaCO₃ 1, 1.1, 2, 2.1, and 3) Possible droughty, Calcium carbonate soils. P, K, and Zn concerns. Potentially high Calcium carbonate may affect availability of P and Zn. Starter fertilizer containing both may be considered for corn..

5. (corn category 2.1) Eroded topsoil with nutrient loss probable. Possible Zn and S deficiencies due to lost organic matter. Insure K is adequate to insure limited water is used efficiently. Erosion risk increases P loss potential. Consider starter fertilizer for corn.

6. (corn category 2.1 & CaCO₃-1, 1.1, 2, 2. 1, and 3) Probable eroded topsoil, Calcium carbonate soils. Possible Zn and S deficiencies due to low organic matter. Insure K is adequate. Erosion risk increases P loss potential. Potentially high Calcium carbonate may affect availability of P and Zn. Starter fertilizer containing both may be considered for corn. If this soil is in the loess region, the loess may have eroded away, exposing high Calcium carbonate glacial till—also called “high Mg soils” or “clay knolls” in that region.

7. (corn categories 2.3 and 2.5) Drought possible/high water table soils. K is needed to strengthen plants from wet and cold disease prone soil in spring and is needed for efficient use of limited water on this porous/droughty soil. This zone also includes steep, eroded soils that seep. All soil supplied plant nutrients are at risk in this situation. These soils may be slow to warm in the spring because of high water tables. Consider P and N in a starter fertilizer to encourage early root development in corn.

8. (corn categories 2.3, and 2.5 and CaCO₃-1, 1.1, 2, 2.2, and 3) Drought possible, high water table, Calcium carbonate soils. Many soil supplied plant nutrients at risk. Insure K is adequate. This zone also includes steep, eroded soils that seep. These soils may be slow to warm in the spring because of high water tables. Also, soils high in Calcium carbonate may affect the availability of P and Zn. For both reasons, a starter containing at least both nutrients may be considered. Consider tiling “wet sand” situations at recommended intervals to reduce soluble salts.

9. (corn category 3) Non-problematic soil characteristics, consistent yielding or high yielding soils. Fertilize accordingly.

10. (corn categories 4 and 5) Probable and possible wet soils. Cool and disease prone. P, K, and Zn concerns. K is needed to strengthen plants in these wet and cold, disease prone soils. P and Zn uptake could be limited in this cool, wet situation. Starter fertilizer containing at least N, P, and Zn should be considered to encourage early root development in corn. Tile at recommended intervals.

11. (Calcium carbonate 2, 2.1, and 3). Possible high Calcium carbonate soils. P and Zn concerns. Possible high CaCO₃ levels could reduce availability of P and Zn. Improving P and Zn soil test levels may be difficult because of possible high Calcium carbonate levels. Starter fertilizer containing at least N, P, and Zn should be considered for corn.

12. (bean category Calcium carbonate 1 and 1.1). High Calcium carbonate soils. P and Zn concerns. High CaCO₃ will reduce availability of P and Zn. Improving P and Zn soil test levels will be difficult because of high Calcium carbonate levels. Starter fertilizer containing at least N, P, and Zn should be considered for corn.

13. (corn categories 4 & 5, CaCO₃-2 & 3) Probable and possible wet soils. Cool and disease prone, possible high Calcium carbonate soils. Many soil supplied plant nutrients at risk. K is needed to strengthen plants in these wet and cool, disease prone soils. P and Zn uptake could be limited in this cool wet situation. Also, possible high Calcium carbonate levels could affect availability of P and Zn. Improving P and Zn soil test levels could be difficult because of possible high Calcium carbonate levels. Starter fertilizer containing at least N, P, and Zn should be considered to encourage early root development in corn. Tile at recommended intervals to remove excess water and reduce soluble salts.

14. (corn categories 4 and 5, CaCO₃-1) Probable and possible wet soils. Cool and disease prone, high Calcium carbonate soils. Many soil supplied plant nutrients at risk. K is needed to strengthen plants in these wet and cool, disease prone soils. P and Zn uptake could be limited in this cool wet situation. Also, high Calcium carbonate levels will affect availability of P and Zn. Improving P and Zn soil test levels will be difficult because of high Calcium carbonate levels. Starter fertilizer containing at least N, P, and Zn should be considered to encourage early root development in corn. Tile at recommended intervals to remove excess water and to reduce soluble salts.

15. Typically not farmed.

### EXAMPLE 2

### Protocol for Determining Seed Placement Categories

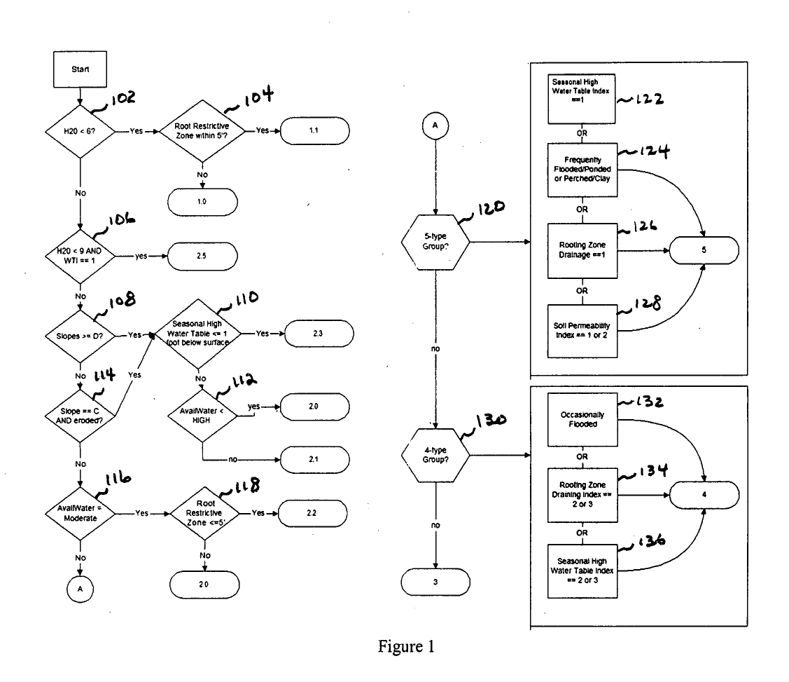

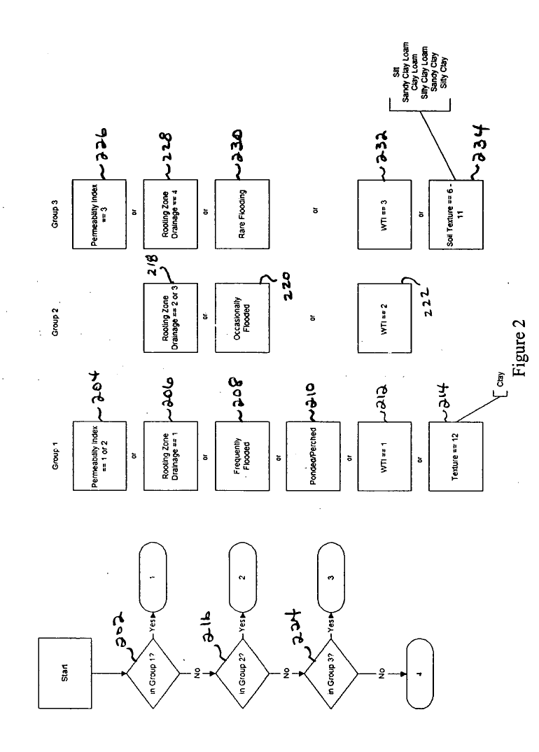

Another embodiment of the present method is disclosed using FIGS. 1 and 2.

Corn:

A flow chart of the logic used in determining Corn Seed Placement Categories is shown in FIG. 1. Initially a decision is made at 102. If the Plant Available Water is less than six inches (i.e., the Available Water Capacity Index is “1” or “2”) and the Root Restrictive Zone (obtained from, e.g., soil survey data) is within five feet of the soil surface, determined at 104, the Seed Placement Category is “1.1.” If the decision at 104 is “no,” the Seed Placement Category is “1.0.” If the Plant Available Water is between 6 inches and 9 inches (i.e., Available Water Capacity Index is “3”) and the Water Table Index is “1,” as determined at 106, the Seed Placement Category is 2.5. If the decisions at 102 and 106 are both negative, and the Slope Index is D, E, or F, as determined at 108, and the Water Table Index is “1” or “2” (e.g., Seasonal High Water Table is one foot, or less, beneath the soil surface), as determined at 110, the Seed Placement Category is 2.3. If the answer to the question posed at 110 is “no” and the Available Water Capacity Index is “1,” “2,” or “3,” as determined at 112, the Seed Placement Category is 2.0. If the answer to the question posed at 112 is “no,” the Seed Placement Category is 2.1. If the answer to the question posed at 108 is “no,” a question is posed at 114 whether the Slope Index is “C” and whether the Eroded Soil Index is “2” or “3.” If the answer is “yes” to the questions posed at 114, the logic returns to the questions posed at 110 and 112, wherein Seed Placement Categories of 2.3, 2.0, and 2.1 are assigned. If the Slope Index is not “C” and the Eroded Soil Index is not “2” or “3,” a question is posed at 116, wherein the Available Water Capacity Index is “3.” If so, the logic flows to whether the Root Restrictive Zone is within 5 feet of the soil surface at 118. If the answer to the query at 118 is positive, the Seed Placement Category is 2.2. If the answer to the query at 118 is negative, the Seed Placement Category is 2.0. If the answers to the questions posed at 102, 106, 108, 114, and 116 are negative, the logic flows to determining whether the Seed Placement Category is “5” at 120. The Seed Placement Category is “5” if 1) the Water Table Index is “1,” as determined at 122; 2) the predominant soils are frequently flooded, ponded or perched as determined at 124; 3) the Rooting Zone Drainage Index is “1,” as determined at 126 or 4) the Soil Permeability Index is “1” or “2,” as determined at 128. If the answer to the query posed at 120 is negative, the logic flows to determine whether the Seed Placement Category is “4,” at 130. The Seed Placement Category is “4” if 1) the field is occasionally flooded at 132; 2) the Natural Root Zone Drainage Index is “2” or “3” as determined at 134; or 3) if the Water Table Index is “2” or “3.” If the answers to the questions posed at 132, 134, and 136 are negative, the Seed Placement Category is “3.”

Soybeans.

Chlorosis.

A protocol for determining the Seed Placement Categories for chlorosis potential of soils may be substantially similar to the protocol described in Example 1, above.

Phytophthora Potential.

An exemplary protocol for determining Phytophthora potential in soybeans is depicted in FIG. 2. In the decision at 202, determination of whether the predominant soil type in a field is in a Seed Placement Category of “1” is made. The Seed Placement Category is “1” if 1) the Soil Permeability Index is “1” or “2” at 204; 2) the Natural Root Zone Drainage Index is “1” at 206; 3) the field is “Frequently Flooded” at 208; 4) the field is ponded or perched at 210; 5) the Water Table Index is “1” at 212 or 6) the Soil Texture Index is 12 at 214. If the queries posed at 204, 206, 208, 210, 212, and 214 are negative, the logic flows to determine whether a Seed Placement Category of “2” is appropriate. A Seed Placement Category of “2” is assigned if 1) the Natural Root Zone Drainage Index is “2” or “3” at 218; 2) the field is “occasionally flooded”) at 220; or 3) the Water Table Index is “2” at 222. If the answers to the questions posed at 218, 220, and 222 are negative, the logic flows to determine whether the predominant soil type in the field merits a Seed Placement Category of “3.” A Seed Placement Category of “3” is assigned if 1) the Soil Permeability Index is “3” at 226; 2) the Natural Root Zone Drainage Index is “4” at 228; 3) the field experiences “rare flooding” at 230; 4) the Water Table Index is “3” at 232; or 5) the Soil Texture Index is between 6-11 at 234. If the answers to the queries at 226, 228, 230, 232, and 234 are negative, the Seed Placement Category is “4.”

Soil complexes require special additional calculations. A map unit may contain two or more soil types in either such an intricate pattern or in so small an area that it is not practical to map the soil types separately. If so, soil complexes are assigned the most yield limiting seed placement category of the two or more soil types in a given complex. Complexes containing more than two soil types require obtaining the answer from the protocol for the first two soils, the comparing the answer to the third soil type in the complex. For complexes with more than three soils, the paired iterations are continued until a final answer is obtained. However, if more than a single factor (e.g., drought and water logging propensity) is present, then both factors must be addressed for proper use and placement. For example, Table 2 shows exemplary complex classifications for corn seed Placement categories. For example, if single soils with Seed Placement Categories with “1” and “5” are present, a complex Seed Placement Category of “2.5” is chosen. Similarly and as illustrated in Table 3, if for example a complex has individual Seed Placement Categories of “1” and “2.1,” a complex Seed Placement Category of “1” is selected. However, if individual Seed Placement Categories of “1.1” and “2” are present, a complex Seed Placement Category of “2” is selected. Referring to Table 4 and with respect to Phytophthora Seed Placement Categories individual Seed Placement Categories of “2” and “3” will result in a complex Seed Placement Category of “2.”

In Table 5, individual Soybean Seed Placement Categories follow the rule stated above. For example, individual Seed Placement Categories of “D/O” and “O/D” result in a complex Seed Placement Category of “D/O.”

TABLE 2
Two Soils in a Complex - Corn Category Placement

| | 1 | 1.1 | 2 | 2.1 | 2.2 | 2.3 | 2.5 | 3 | 4 | 5 | 6 |
|---|---|---|---|---|---|---|---|---|---|---|---|
| 1 | 1 | 1.1 | 1 | 1 | 1 | 2.3 | 2.5 | 1 | 2.5 | 2.5 | 1 |
| 1.1 | 1.1 | 1.1 | 1.1 | 1.1 | 1.1 | 2.3 | 2.5 | 1.1 | 2.5 | 2.5 | 1.1 |
| 2 | 1 | 1.1 | 2 | 2 | 2.2 | 2.3 | 2.5 | 2 | 2.5 | 2.5 | 2 |
| 2.1 | 1 | 1.1 | 2 | 2.1 | 2.2 | 2.3 | 2.5 | 2.1 | 2.3 | 2.3 | 2.1 |
| 2.2 | 1 | 1.1 | 2.2 | 2.2 | 2.2 | 2.3 | 2.5 | 2.2 | 2.5 | 2.5 | 2.2 |
| 2.3 | 2.3 | 2.3 | 2.3 | 2.3 | 2.3 | 2.3 | 2.3 | 2.3 | 2.3 | 2.3 | 2.3 |
| 2.5 | 2.5 | 2.5 | 2.5 | 2.5 | 2.5 | 2.3 | 2.5 | 2.5 | 2.5 | 2.5 | 2.5 |
| 3 | 1 | 1.1 | 2 | 2.1 | 2.2 | 2.3 | 2.5 | 3 | 4 | 5 | 3 |
| 4 | 2.5 | 2.5 | 2.5 | 2.5 | 2.5 | 2.3 | 2.5 | 4 | 4 | 5 | 4 |
| 5 | 2.5 | 2.5 | 2.5 | 2.5 | 2.5 | 2.3 | 2.5 | 5 | 5 | 5 | 5 |
| 6 | 1 | 1.1 | 2 | 2.1 | 2.2 | 2.3 | 2.5 | 3 | 4 | 5 | 6 |

TABLE 3
Two Soils in a Complex - Soybeans - Calcium Carbonate Index

| | 1 | 1.1 | 2 | 2.1 | 3 | 4 | 6 |
|---|---|---|---|---|---|---|---|
| 1 | 1 | 1 | 1 | 1 | 1 | 1 | 1 |
| 1.1 | 1 | 1.1 | 2 | 1.1 | 1.1 | 1.1 | 1.1 |
| 2 | 1 | 2 | 2 | 2 | 2 | 2 | 2 |
| 2.1 | 1 | 1.1 | 2 | 2.1 | 2.1 | 2.1 | 2.1 |
| 3 | 1 | 1.1 | 2 | 2.1 | 3 | 3 | 3 |
| 4 | 1 | 1.1 | 2 | 2.1 | 3 | 4 | 4 |
| 6 | 1 | 1.1 | 2 | 2.1 | 3 | 4 | 6 |

TABLE 4
Two Soils in a Complex - Soybeans - Phytophthora Potential

| | 1 | 2 | 3 | 4 |
|---|---|---|---|---|
| 1 | 1 | 1 | 1 | 1 |
| 2 | 1 | 2 | 2 | 2 |
| 3 | 1 | 2 | 3 | 3 |
| 4 | 1 | 2 | 3 | 4 |

TABLE 5
Two Soils in a Complex - Soybean Seed Category

| | O | O/D | D/O | D |
|---|---|---|---|---|
| O | O | O/D | D/O | D |
| O/D | O/D | O/D | D/O | D |
| D/O | D/O | D/O | D/O | D |
| D | D | D | D | D |

Because more than one of the present management categories is often present in a single field, may be advantageous to plant separate sets of crop varieties, adapted to each of the management categories, in the field. Alternatively, a set of crop varieties could be chosen that is best adapted to the most prevalent management category in the field or to the most problematic management category in the field.

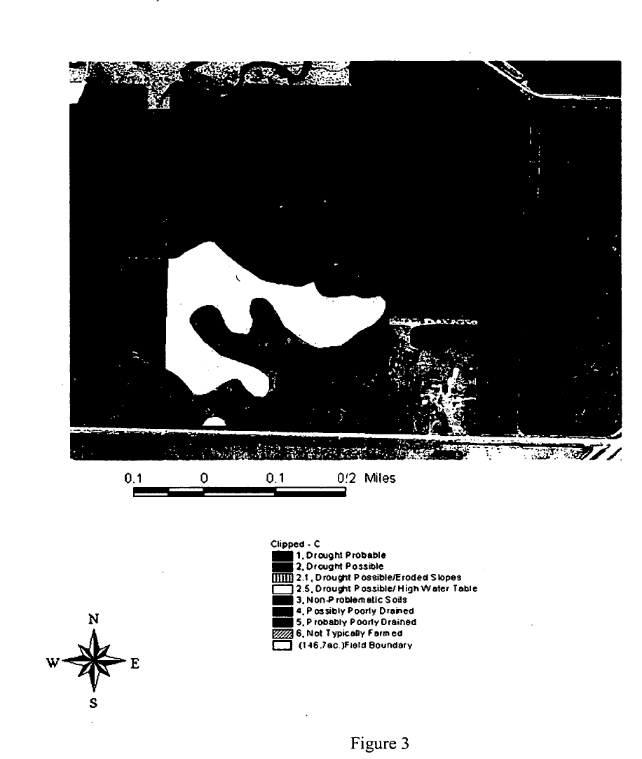

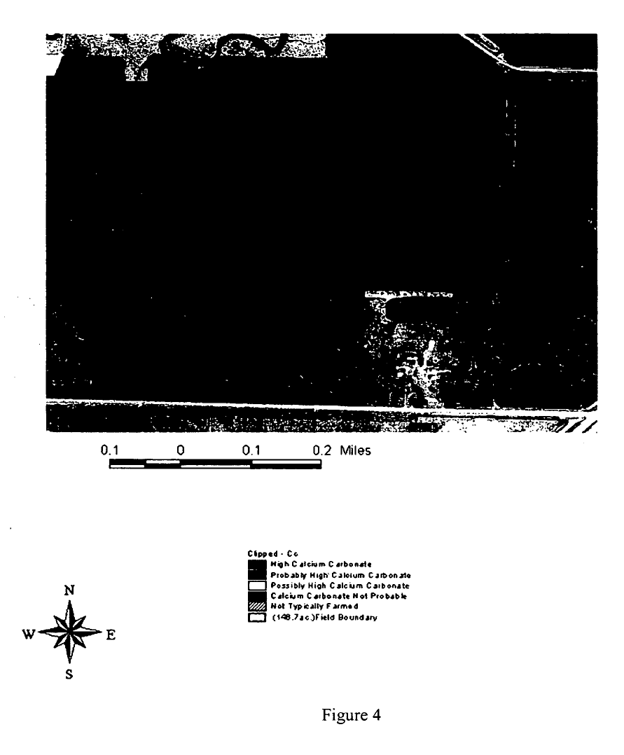

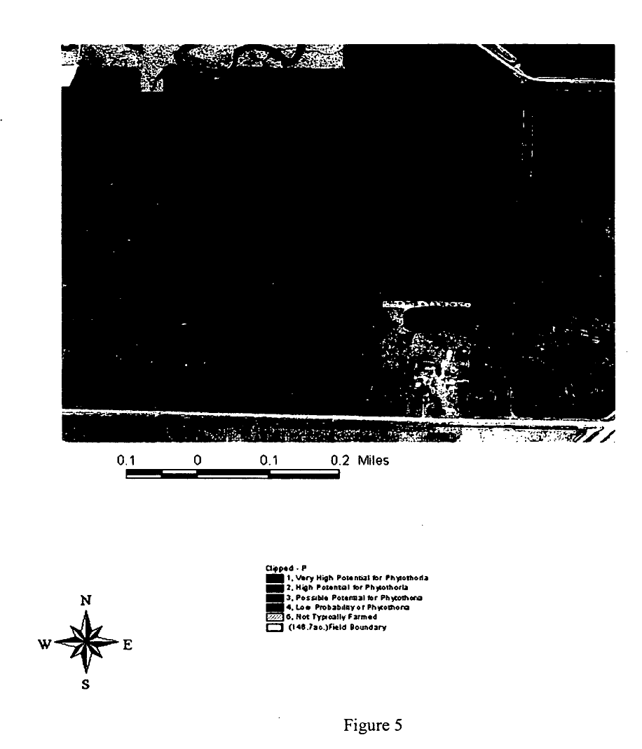

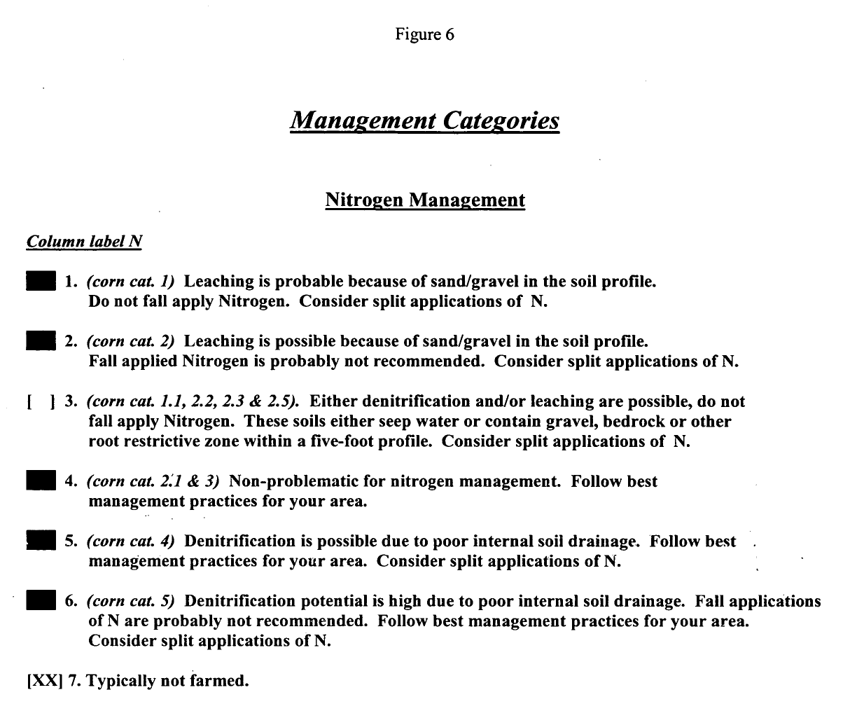

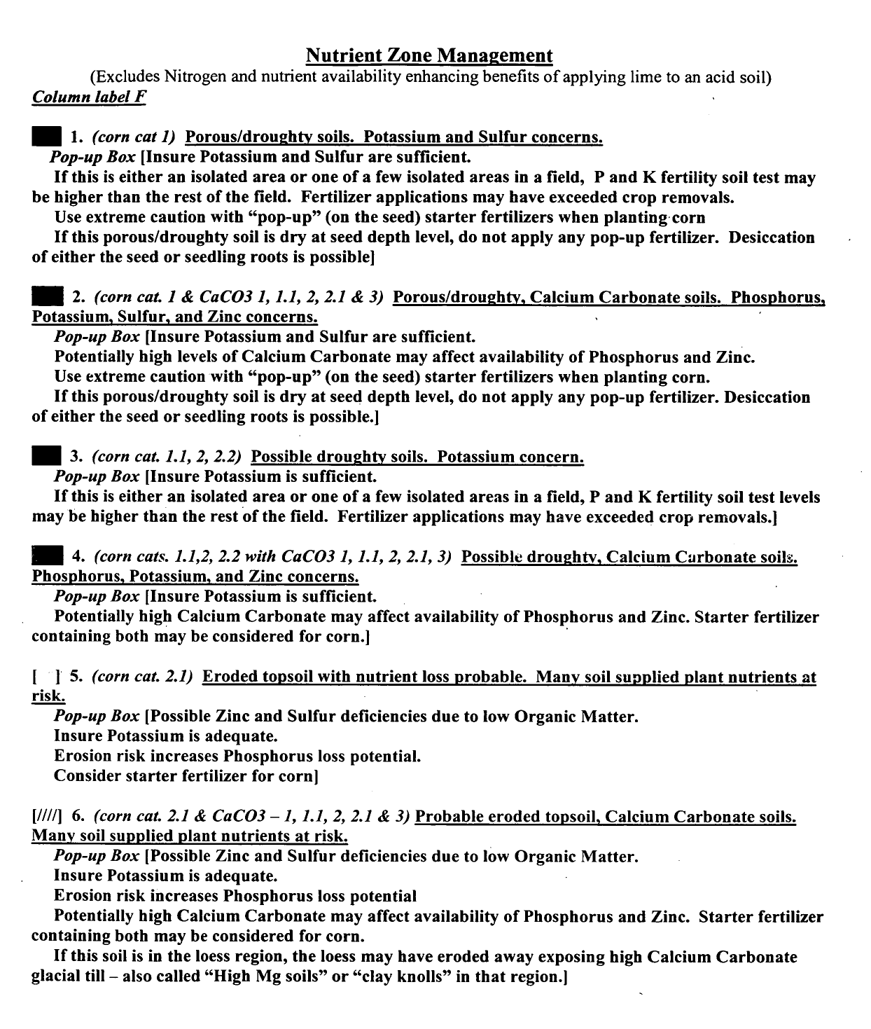

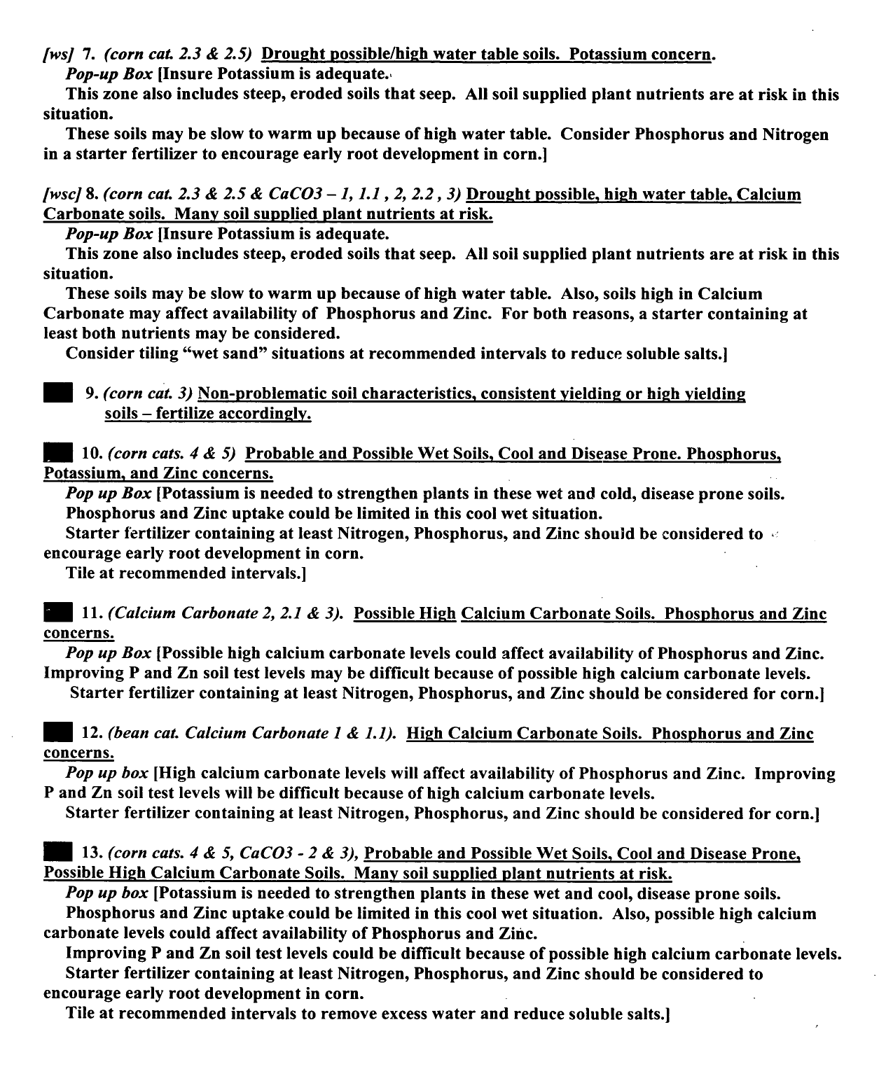

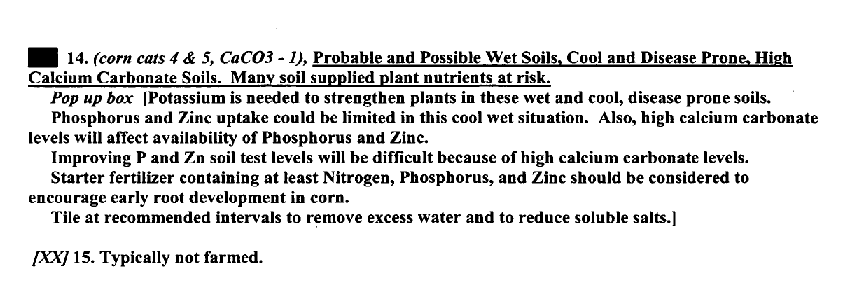

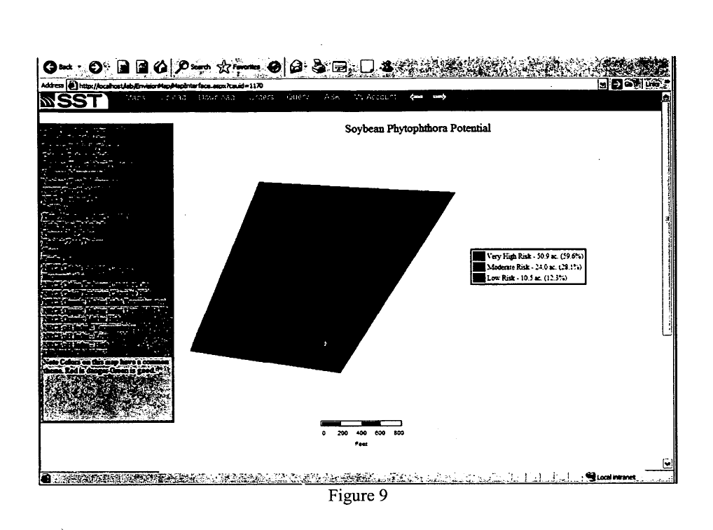

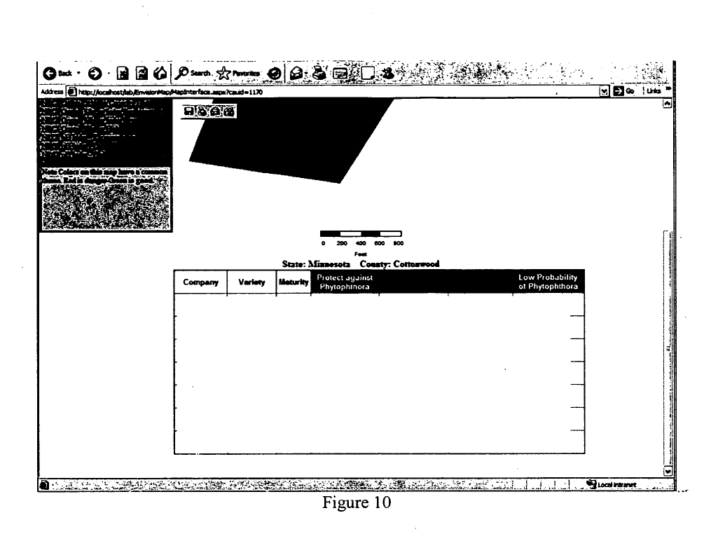

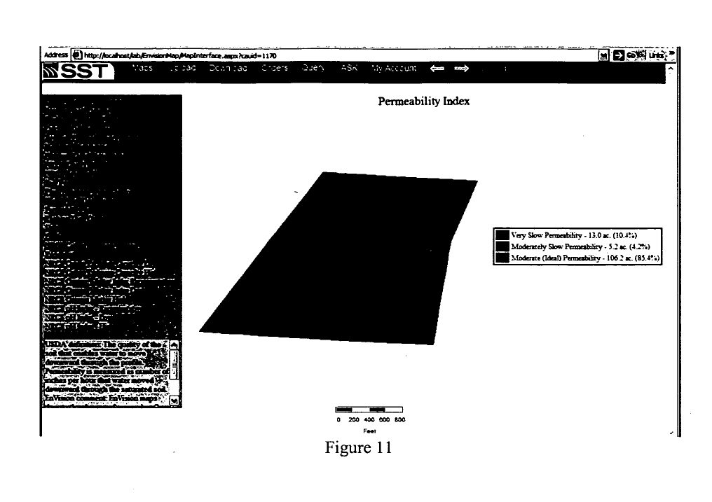

After the present soil management categories have been determined, they are depicted, e.g., overlaid on a digitized soil map on a computer screen. FIG. 3 depicts a digitized soil map which is overlaid with the present soil management categories shown as various colors for corn. FIG. 4 shows a digitized soil map which is overlaid with the present soil management categories shown as colors for soybean iron chlorosis. FIG. 5 depicts a digitized soil map which is overlaid with the present soil management categories for Phytophthora/fungi risk. FIG. 6 is a representation of indicia (e.g., colors) used for recommendations for management of nitrogen and for Nutrient Zones for phosphorous, potassium, zinc, and sulfur. FIGS. 7 and 8 are respective and exemplary, color-coded depictions of the present management categories for corn and soybean varieties, wherein the colors used are those of corresponding soil management categories. Using programs, e.g., following the logic of Example 2, below, Permeability Indices may be depicted in a screen as shown in FIG. 9; recommended crop cultivars, e.g., soybean varieties may be shown for a field in which the instant Soybean Phytophthora Seed Placement Categories are depicted as shown in FIG. 10; or the areas of a field may be displayed in terms of the present Soybean Phytophthora Seed Placement Categories as depicted in FIG. 11. Any suitable programming language (e.g., FORTRAN, COBOL) may be used in specific applications.

Because numerous modifications of this invention may be made without departing from the spirit thereof, the scope of the invention is not to be limited to the embodiments illustrated and described. Rather, the scope of the invention is to be determined by the appended claims and their equivalents.

1. A process of selecting a crop variety, comprising:
viewing a digitized map of a field, the digitized map depicting areas denoted by an indicium, said areas having an optimized adaptation for a variety subset; and
selecting the crop variety from the variety subset, the variety subset denoted by the indicium.

2. The process of claim 1, in which the indicium includes a color.

3. The process of claim 1, in which the crop variety is a corn variety or a soybean variety.

4. The process of claim 1, in which said indicium is a classification at least partially based on physical properties of soils present in the field.

5. The process of claim 4, in which the physical characteristic includes slope, erosion, soil water holding capacity, soil texture, pH, permeability, seasonal presence of saturated soil layers, organic matter, or cation exchange capacity.

6. The process of claim 5, in which the crop variety is a soybean variety and in which the indicium indicates Phytophthora or iron chlorosis predisposition in the digitized map and Phytophthora or iron chlorosis tolerance in the soybean variety.

7. The process of claim 5, in which the crop variety is a corn variety and in which the indicium indicates drought predisposition in the digitized map and drought tolerance in the corn variety.

8. A process of integrating a set of soil characteristics and a set of crop variety characteristics, comprising:
obtaining a set of physical properties characterizing a subset of soil types;
using the set of physical properties to obtain a set of corresponding indices;
using the set of indices to obtain a set of management categories, each of the soil types characterized by one of the management categories;
obtaining a set of adaptation characteristics for a corresponding set of crop varieties; and
using the set of adaptation characteristics of each of the set of crop varieties to designate which of the set of crop varieties should be planted in each of the management categories.

9. The process of claim 8, further comprising assigning an indicium to each of the set of management categories.

10. The process of claim 9, in which the indicium includes a color.

11. The process of claim 8, in which the crop varieties are corn varieties or soybean varieties.

12. The process of claim 8, in which the physical properties include slope, erosion, soil water holding capacity, soil texture, pH, permeability, seasonal presence of saturated soil layers, organic matter, or cation exchange capacity.

13. The process of claim 8, in which the crop variety is a soybean variety and in which the indicium indicates Phytophthora or iron chlorosis predisposition in the digitized map and Phytophthora or iron chlorosis tolerance in the soybean variety.

14. The process of claim 8, in which the crop variety is a corn variety and in which the indicium indicates drought predisposition in the digitized map and drought tolerance in the corn variety.

15. A process of determining crop management categories, comprising:
compiling a set of physical properties for a set of soil types in a region; and
assigning each of the soil types in the region to one of the crop management categories.

16. The process of claim 15, in which the physical properties include slope, erosion, soil water holding capacity, soil texture, pH, permeability, seasonal presence of saturated soil layers, organic matter, or cation exchange capacity.

17. The process of claim 15, in which the crop management categories are indices grouping one or more of the set of soil types according to how crop plants grow and develop thereon.

18. The process of claim 17, in which the crop plants are corn plants or soybean plants.

19. The process of claim 15, further comprising a unique indicium to each of the crop management categories.

20. The process of claim 19, in which the indicium includes a color.

21. A process of determining seeding rate of a variety being seeded, comprising:
providing a planter with a variable seeding rate, the planter in electrical or electromagnetic communication with a digitized soil map, the digitized soil map having areas defined by crop management categories; and
seeding the variety, the seeding rate of the variety determined by the position on the planter relative to the crop management categories.

22. An integrated data set, comprising:
categories describing corresponding sectors of a field and derived from soil indices, each of said categories describing one of said corresponding sectors of the field having a unique indicium; and
categories describing corresponding sets of crop varieties and depicting the response of the crop variety set to the soil and environmental indices, each of the categories describing a crop corresponding to the categories describing the sector of the field and having the unique indicium.

23. The data set of claim 22, in which one of the categories describing the sector of the field are further determined by an impervious soil layer present in the sector of the field.

24. The data set of claim 22, in which the crop variety is a corn variety.

25. The data set of claim 24, in which the categories describing a sector of the field describe the propensity of the sector to experience a water shortage during a growing season.

26. The data set of claim 24, in which the categories describing a sector of the field describe the propensity of the sector to experience water excess during a growing season.

27. The data set of claim 22, in which the crop variety is a soybean variety.

28. The data set of claim 27, in which the categories describing a sector of the field describe the propensity of the sector to promote growth and development of Phytophthora during the growing season.

29. The data set of claim 27, in which the categories describing a sector of the field describe the propensity of the sector to induce iron chlorosis during the growing season.

30. The data set of claim 22, in which the indicium can be graphically depicted as a color.
